SEQUENCE LISTING (6 sequences)

SEQ ID NO: 1 - protein, 11 aa, Artificial sequence
  peptide motif
  misc_feature (9)..(9): Xaa can be any naturally occurring amino acid
ACDCRGDIXFC

SEQ ID NO: 2 - protein, 14 aa, Artificial sequence
  peptide motif
KLAKLAKKLAKLAK

SEQ ID NO: 3 - DNA, 22 bp, Artificial sequence
  Primer
gaccctcggacaactgtgtctt

SEQ ID NO: 4 - DNA, 21 bp, Artificial sequence
  Primer
gcgttgtagaagcaacagtga

SEQ ID NO: 5 - DNA, 20 bp, Artificial sequence
  Primer
tggcacagtcaaggctgaga

SEQ ID NO: 6 - DNA, 20 bp, Artificial sequence
  Primer
cutctgagtggcagtgatgg

(19) United States
(12) Patent Application Publication
(10) Pub. No.: US 2022/0079889 A1
(43) Pub. Date: Mar. 17, 2022

(54) USE OF CANNABANOIDS IN THE TREATMENT OF PROLIFERATIVE DIABETIC RETINOPATHY

(71) Applicant: VERDANT PHARMACEUTICALS, LLC, PARK CITY, UT (US)

(72) Inventor: Dipak PANIGRAHI, San Clemente, CA (US)

(21) Appl. No.: 17/194,087

(22) Filed: Mar. 5, 2021

Related U.S. Application Data
(60) Provisional application No. 62/985,782, filed on Mar. 5, 2020.

(51) Int. Cl. A61K 31/05; A61K 38/28; A61K 31/58; A61K 31/64; A61K 31/155; A61K 31/426; A61K 31/4439; A61K 39/395; A61K 31/506; A61K 31/44; A61K 31/404; A61K 38/17; A61K 47/68; A61K 31/409; A61K 47/44; A61K 47/10; A61K 47/26; A61K 41/00; A61P 27/02

(52) U.S. Cl. CPC A61K 31/05; A61P 27/02; A61K 31/58; A61K 31/64; A61K 31/155; A61K 31/426; A61K 31/4439; A61K 39/3955; A61K 31/506; A61K 31/44; A61K 31/404; A61K 38/179; A61K 47/6811; A61K 31/409; A61K 47/44; A61K 47/10; A61K 47/26; A61K 41/0071; A61K 38/28

(57) ABSTRACT

Cannabidiol (CBD) based compositions for use in the treatment of angiogenesis in proliferative diabetic retinopathy (PDR) is provided. The invention also relates to methods and compositions for treating conditions associated with lymphangiogenesis using cannabidiol (CBD). Preferably the angiogenesis manifest as abnormal vascular lesions with visual impairment and the angiogenesis is treatment resistant.

### CROSS-REFERENCE TO RELATED APPLICATIONS

The present application claims the benefit of U.S. Provisional Application No. 62/985,782, filed Mar. 5, 2020, the content of which is incorporated by reference in its entirety.

### FIELD OF THE INVENTION

Provided herein are methods and compositions for treating conditions associated with lymphangiogenesis using cannabidiol.

### BACKGROUND

Diabetic Retinopathy (DR) is the leading cause of vision loss in adults aged 20-74 years (Klein, R., et al. Diabetes in America (1995) National Institute of Health, 293-338). From 1990-2010, DR ranked as the fifth most common cause of preventable blindness and fifth most common cause of moderate to severe visual impairment (Tien Yin Wong, et al. Am J Ophthalmol. (2006) March; 141(3): 446-455). In 2010, of an estimated 285 million people worldwide with diabetes, over one-third had signs of DR, and a third of these were afflicted with vision-threatening diabetic retinopathy (VTDR), defined as severe non-proliferative DR or proliferative DR (PDR) and/or the presence of diabetic macular edema (DME) (Ryan Lee, et al. Eye Vis (London). (2015); 2: 17) These estimates are expected to rise further due to the increasing prevalence of diabetes, generalized aging of the population, and/or increasing life expectancy of those with diabetes.

PDR represents the most common vision-threatening pathology particularly among patients with type I diabetes. However, DME is responsible for most of the visual loss experienced by patients with diabetes as it remains the major cause of vision loss in the highly prevalent type 2 diabetes and is invariably present in patients with type 2 diabetes with PDR. In addition to vision loss, DR and DME have also been shown to contribute to the development of other diabetes-related complications including nephropathy, peripheral neuropathy and cardiovascular events.

Globally, the most clinically important risk factors for progression to vision loss include duration of diabetes, glycemic status, and hypertension. Control of serum glucose and blood pressure have been shown to be effective in preventing vision loss due to DR. Prevalence and risk factors of DR have been studied widely in previous studies (see Table 1).

TABLE I
Age-standardized prevalence of DR in diabetic subjects aged 20-79
years, using studies with similar methodologies and ophthalmologic
definitions (from Yau et al. (2012) Diabetes Care, 35(3): 556-564.

| | Studies | | | Age-standardized |
|---|---|---|---|---|
| | included | Total | | prevalence per 100 |
| Overall | (n) | (N) | Cases (n) | (95% CI) |
| Any DR | 18 | 12,620 | 4,487 | 35.36 (35.17-35.56) |
| PDR | 21 | 13,436 | 957 | 7.24 (7.15-7.33) |
| DME | 20 | 14,554 | 1,039 | 7.48 (7.39-7.57) |
| VTDR | 18 | 12,710 | 1,481 | 11.72 (11.61 = 11.83) |
| Men | | | | |
| Any DR | 18 | 6,252 | 2,263 | 36.27 (35.99-36.55) |
| PDR | 21 | 6,376 | 469 | 7.53 (7.39-7.66) |
| DME | 20 | 7,010 | 486 | 7.44 (7.30-7.57) |
| VTDR | 18 | 6,051 | 704 | 11.74 (11.57-11.90) |
| Women | | | | |
| Any DR | 18 | 6,368 | 2224 | 34.46 (34.19-34.73) |
| PDR | 21 | 7,060 | 488 | 6.98 (6.86-7.10) |
| DME | 20 | 7,544 | 553 | 7.54 (7.42-7.66) |
| VTDR | 18 | 6,659 | 777 | 11.70 (11.55-11.86) |

Similarly there are studies that have evaluated the regional and ethnic differences in diabetes. Despite these studies, the overall epidemiological data on DME remains relatively scarce. A review conducted in 2012 suggested that up to 7% of people with diabetes may have DME and a correlation of risk factors to DR has been demonstrated. Recently, new information on the epidemiology of DR and DME has been published from both developed and developing countries. A recent systematic review estimated that in 2010, 3.63 million people worldwide suffer from moderate and severe vision loss due to DR and its related sequelae, defined as visual acuity in the better eye being worse than Snellen 6/18 but at least 3/60. An estimated 850.000 more people suffer from DR-related blindness, defined as visual acuity worse than 3/60 in the better eye. Prevalence of vision impairment and blindness due to DR was found to be trending upwards, even though the prevalence of vision impairment and blindness was decreasing. Findings from reviews of cross-sectional studies in Europe, South-East Asia, and Oceania consistently show DR to be the fifth most common cause of moderate and severe vision loss and blindness (behind other causes such as uncorrected refractive error, cataracts, macular degeneration, and glaucoma) in these regions. In Africa, DR is the sixth most common cause of visual impairment and blindness, behind the above-listed conditions and trachoma (an ocular infection caused by the bacterium Chlamydia trachomatis that can lead to blindness that is currently the leading cause of preventable blindness worldwide). In the USA, the WESDR investigated visual impairment in patients with type 1 diabetes, and found that 25-year cumulative incidence of visual impairment (defined as poorer than 6/12 best-corrected visual acuity in the better eye) and severe visual impairment (defined as poorer than 6/60 best-corrected visual acuity in the better eye) to be 13 and 3%, respectively. Recent data from Leeds, UK, found that in 2008 to 2010, DR accounted for 6.1-8.3% of visual impairment certification. Extrapolated to the total population of the metropolitan area in Leeds, estimates suggest that as many as 30.0 to 43.2 people per million per year will become severely visually impaired due to DR and its sequelae. In Fife, Scotland, between 2000 and 2009, the mean incidence of blindness (defined as above) was 13.8 per million per year for the total population of the county. In the Sankara Nethralaya Diabetic Retinopathy Epidemiology and Molecular Genetics Study (SN-DREAMS) (a study drawing from rural areas of India) in type 2 diabetes, the prevalence of visual impairment and blindness was 4 and 0.1%, respectively. In most studies, DME was defined by hard exudates in the presence of microaneurysms and blot hemorrhages within one disc diameter of the foveal center. Clinically significant macular edema (CSME) sits in the more severe spectrum of DME and is defined by the presence of edema within 500 μm of the foveal center, or focal photocoagulation scars present in the macular area. The prevalence of DME among recent cross-sectional studies is summarized in Table 2. XC

TABLE 2
Prevalence and Severity of Diabetic Retinopathy and Macular Edema

| | Total | | | | | |
|---|---|---|---|---|---|---|
| | Diabetes | | | | | |
| | Sample | White | Black | Hispanic | Chinese | |
| | (N = 778), | (N = 153), | (N = 235), | (N = 235), | (N = 101), | P |
| Characteristic | n (%) | n (%) | n (%) | n (%) | n (%) | Value* |
| No Retinopathy | 520 (66.8) | 115 | 183 (63.3) | 147 (62.6) | 75 (74.3) | |
| | | (75.2) | | | | |
| Retinopathy | 258 (33.2) | 38 (24.8) | 106 (36.7) | 88 (37.4) | 26 (25.7) | .01 |
| Minimal | 116 (14.9) | 22 (14.4) | 48 (16.6) | 37 (15.7) | 9 (8.9) | |
| Early-moderate | 112 (14.4) | 12 (7.8) | 46 (15.9) | 42 (17.9) | 12 (11.9) | |
| Severe- | 30 (3.9) | 4 (2.6) | 12 (4.2) | 9 (3.8) | 5 (5.0) | |
| proliferative | | | | | | |
| No macular edema | 694 (91.0) | 146 | 248 (88.9) | 208 (89.3) | 92 (91.1) | |
| | | (97.3) | | | | |
| Macular edema | 69 (9.0) | 4 (2.7) | 31 (11.1) | 25 (10.7) | 9 (8.9) | .007 |
| present | | | | | | |
| CSME | 43 (5.6) | 3 (2.0) | 21 (7.5) | 16 (6.9) | 3 (3.0) | |
| Vision-threatening | 60 (7.9) | 7 (4.5) | 26 (9.3) | 20 (8.7) | 7 (6.9) | .28 |
| retinopathy | | | | | | |

Among the population-based studies, prevalence of DME among patients with type 1 diabetes was between 4.2 and 7.9° %. In patients with type 2 diabetes, it was between 1.4 and 12.8% i. Non-stereoscopic fundus photography was used in most studies, which affects the accuracy of DME assessment. About half of the studies defined macular edema using the CSME criteria, and hence only the more severe spectrum of DME was captured in these studies meaning that the true numbers may be much higher. Overall, the heterogeneity in methodology causes comparison of prevalence between these studies to be a challenge. The prevalence of DME among patients with diabetes is generally much lower than that of DR. Of note, there does not appear to be observable difference between prevalence of DME between Western or Eastern populations.

Blood vessels generally supply oxygen and/or nutrients to and remove waste products, gasses (such as CO2), and/or various solutes from biological tissue (a cellular organizational level between cells and a complete organ/organ system.) A tissue is an ensemble of similar cells and their extracellular matrix from the same origin that together carry out a specific function. Organs are then formed by the functional grouping together of multiple tissues. Angiogenesis refers to the biological process in which blood vessels are formed. Angiogenesis is an essential part of many biological processes (for example, reproduction, embryonic development, and wound repair). Angiogenesis however, normally occurs in humans and animals in a very limited and well controlled set of circumstances.

Angiogenesis and the rate of angiogenesis involve changes in the local equilibrium between positive and negative regulators on the growth of microvessels. Abnormal angiogenesis occurs when the body loses at least some control of this equilibrium, resulting in either excessive and/or insufficient blood vessel growth solely or in combination. For example, the absence of angiogenesis normally required for natural healing conditions can lead to conditions such as ulcers, strokes, and heart attacks. In contrast, excessive blood vessel proliferation has been associated with many cancers, tumor growth, tumor spread (metastasis), psoriasis, rheumatoid arthritis. Altered angiogenesis underlies many conditions in the eye and is associated with ocular neovascularization, such as corneal neovascularization, choroidal neovascularization (as can be seen in wet age related macular degeneration), and PDR (proliferative diabetic retinopathy).

There are also instances in which a greater degree of angiogenesis is desirable clinically/therapeutically such as increasing blood generalized circulation, modulating (up and/or down) wound healing, and/or facilitating ulcer resolution (lowering infection and promoting healing). For example, researchers have investigated the use of recombinant angiogenic growth factors, such as the fibroblast growth factor (FGF) family, endothelial cell growth factor (ECGF) family, and more recently, vascular endothelial growth factor (VEGF) family to induce collateral artery development in animal models of myocardial and hind limb ischemia.

In contrast to excessive neovascularization, there are many instances in which inhibition of angiogenesis and/or regression of blood vessels is desirable. For example, many diseases are driven by persistent unregulated angiogenesis (often referred to as neovascularization). Many solid tumors are vascularized as a result of angiogenesis such that the neovascularization provides the tumors with a sufficient supply of oxygen and nutrients that permit them to grow rapidly and metastasize (spread/move/travel). Thus, tumor growth and metastasis can be angiogenesis-dependent A tumor must continuously stimulate the growth of capillary blood vessels for the tumor itself to grow. Another example is in arthritis, where capillary blood vessels invade the joint and destroy cartilage resulting in abnormal function. Of note, in the eyes, diabetes can induce capillaries to invade the choroid, retina, vitreous body, iris, and/or the ocular angle (in the anterior chamber between the iris plane and the overlying cornea) These blood vessels may be very fragile causing bleeding into the eye resulting in various sequelae such as physical obstruction (of intraocular orifices/spaces and/or the visual pathway), blood vessel blockage, traction (fibrosis), decreased vision, the presence of “floaters” (free floating blood/blood products with resulting fibrosis forming bodies within the vitreous body or directly in the visual axis), hypoxia (decreased oxygen supply) to the retina, macula, choroid, iris/iris root, cornea (any level including the endothelium, basement membrane, intervening medial layers, and surface epithelium), decreased/alterations in composition of the tear film (via compositional changes involving lipid, protein, and other constituents of the normal tear film), altered/decreased tear production (via impact on those cells/tissues/glands involved in the production/secretion of tears or in alterations to the normal tear production reflex driven in part by afferent and/or efferent nerves), altered and/or decreased corneal neural sensitivity (neuropathy), altered/decreased contrast sensitivity, altered/decreased dark adaptation (visual cycle processes, coordination, and/or control), abnormal ocular reflexes (such as pupillary change in response to light, altered sympathetic/parasympathetic responses), increased intraocular pressure, retinal swelling (including diabetic macular edema), intraretinal bleeding, vitreoretinal traction (including internal limiting membrane ILM traction, vitreoretinal traction, and/or vitreomacular traction (VMT)), vitreoretinal fibrosis, retinal tears (partial and/or complete), retinal detachment (partial and/or full) all of which can ultimately cause visual disability and/or blindness.

In ocular disorders, neovascularization is the most common cause of blindness. One form of ocular neovascularization is corneal neovascularization. Corneal neovascularization is associated with excessive blood vessel ingrowth into the cornea from the limbal vascular plexus. Since the cornea normally is devoid of blood and lymphatic vessels, oxygen supply to the cornea normally is supplied from the air via diffusion through the tear film. When the normal supply of oxygen from the air-to-tear film-to-cornea is altered (for example by use of contact lenses), the local equilibrium between positive and negative regulators that controls growth of microvessels can shift to favor neovascularization of the cornea Severe cases of corneal neovascularization can result in severe visual disability and/or blindness.

Another form of ocular neovascularization is choroidal neovascularization (CNV) which can take multiple forms (as an example retinal angiomatous proliferation RAP). Choroidal neovascularization can lead to hemorrhage and fibrosis, with resulting visual loss in a number of eye conditions (for example, age-related macular degeneration, ocular histoplasmosis syndrome, pathologic myopia, angioid streaks, idiopathic disorders, choroiditis, choroidal rupture, overlying choroid nevi, and certain inflammatory diseases). One of the disorders, namely, age-related macular degeneration (AMD), is the leading cause of severe vision loss in people aged 65 and above (Bressler, et al. (1988) Surv. Ophthalmol. 32, 375-413, Guyer, et al. (1986) Arch. Ophthalmol. 104, 702-705, Hyman, et al. (1983) Am. J. Epidemiol. 188, 816-824, Klein & Klein (1982) Arch. Ophthalmol. 100, 571-573, and Leibowitz, et al. (1980) Surv. Ophthalmol. 24, 335-610).

Although clinico-pathologic research has made progress into the understanding of ocular angiogenesis and neovascularization, little is understood about the true etiology and pathogenic role they play in diseases of the eye (such as diabetic retinopathy, macular degeneration, corneal neovascularization, iris neovascularization and ocular angle neovascularization.

In the eye, diabetic retinopathy is broadly classified into two varieties based on morphology, non-proliferative diabetic retinopathy (NPDR) and proliferative diabetic retinopathy (PDR) (though various morphologies/combinations may be found in between these broad classifications making absolute classification difficult. Further, fluctuating glycemic states can induce rapid changes between the two categories). Whilst the condition usually first manifests as NPDR, multiple factors such as poor glycemic control (shifts between high and low blood sugar concentrations or globally inadequate control of blood sugar concentrations), dyslipidemia (alterations in physiologic lipids, oils and/or fatty acids (either monomers bonded together as polymers or esters)), and hypertension (blood pressure elevated above physiological normal parameters in either systolic and/or diastolic phases) can promote the conversion of NPDR to PDR (Yau, et al. (2012) Diabetes Care, March; 35(3): 556-564) While NPDR can have its own visual sequelae, it seldom results in total blindness instead causing alterations/decreases in vision (functional changes, measurable changes, and/or neurosensory changes such as those that occur from modification of supportive cells and/or tissue such as neurons and/or glial cells) either alone and/or through alternative and/or related mechanisms like diabetic macular edema (DME) (Zhang, et al. (2014) Cell Biosci., 4:27, Klein, et al (2007) Ophthalmic Epidemiol. 14, 179-183, Klein, et al. (1984) Arch Ophthalmol, 102:527-532, Klein, et al. (1992) Ophthalmology, 99:58-62, The Diabetes Control and Complications Trial Research Group (1993) N Engl J Med, 329:977-986) Historically, the earliest forms of diabetic retinopathy have been managed either through lifestyle modifications, medications (topical, injected, device eluting), and, in recalcitrant cases, laser therapy such as pan-retinal photocoagulation (PRP). Any sequence and/or combination of these interventions/treatments may be utilized in treating diabetic retinopathy.

DR prevention and/or control of the metabolic abnormalities can have a substantial effect on the development of diabetic microvascular complications. Both the Diabetes Control and Complications Trial and the U.K. Prospective Diabetes Study demonstrated that optimal metabolic control reduces the incidence and progression of DR and that the benefits of intensive glycemic control could persist over an extended period of follow-up. Based on research, there is no question that optimal metabolic control should be an important treatment goal that is implemented early and maintained for as long as is safely possible. In studies, the rigid control of hypertension had also been demonstrated as being effective in reducing disease progression. In DR, hyper-lipidemia has been linked to the presence of retinal hard exudates in patients and there is some evidence that lipid-lowering therapy may reduce DR pathology (hard exudates and or microaneurysms).

Staying within the recommended range of HbA1c (6.5-7.0%), (Hemoglobin A1c, often abbreviated HbA1c, is a form of hemoglobin (a blood pigment that carries oxygen) that is bound to glucose. The blood test for HbA1c level is routinely performed in people with type 1 and type 2 diabetes mellitus to provide insight into glycemic control summated over a period of 3-4 months), blood pressure (130/85), and/or low density lipoprotein (LDL) cholesterol (100 mg/dl). However, many patients fail to achieve or maintain these levels of metabolic control as ideal control is difficult to achieve and is not without risks. For example, in patients who do achieve a significant reduction in HbA1c remaining within the expected physiological range that demonstrates tight control, there is an associated increased risk of severe hypoglycemia. This is but a single finding highlighting the difficulties in optimal glycemic control for diabetic patients.

In the eye, once sight-threatening DR has been detected, treatment options other than those aimed at treating diabetes generally (glycemic controls, lipid homeostasis, and control of blood pressure) are somewhat limited. The device-based interventional treatments are called laser photocoagulation and pars plana vitrectomy (PPV). In large, well-controlled studies, laser photocoagulation therapy has repeatedly been proven to be effective in reducing DR progression. Laser photocoagulation is used to treat both DR and DME. The goal of macular laser photocoagulation for DME is to limit vascular leakage through a series of focal laser burns at leaking microaneurysms or grid laser burns in regions of diffuse breakdown of the blood-retinal barrier that are associated with condition. By ablating ischemic areas of the peripheral retina, the stimulus for the release of angiogenic growth factors is decreased. Results of the Diabetic Retinopathy Study (DRS) demonstrated that PRP laser effectively reduces the risk of vision loss in the majority (60%) of patients with PDR. The ETDRS compared outcomes in eyes assigned to either deferral of macular laser photocoagulation versus immediate treatment in those diabetic patients diagnosed with clinically significant DME. Results demonstrated that macular laser photocoagulation reduced the risk of vision loss by 50% for patients with clinically significant DME. Repeated studies demonstrate that globally, macular focal and grid laser photocoagulation is clearly indicated for the treatment of clinically significant DME.

After preventive measures have been exhausted and the therapeutic benefits afforded through laser treatment are exhausted, surgical pars plana vitrectomy (PPV) can, in many cases, prevent severe vision loss in patients with advanced stages of DR. PPV is a subtractive process in which the native tissue within the center of the eye (vitreous body) is removed mechanically and replaced with either physiologically buffered saline or in extreme cases, silicone oil During vitrectomy, incisions are made at the pars plana, a portion of the sclera located posterior to the cornea and lens but anterior to the retina. The procedure may also be used to release vitreoretinal traction by excising membranes causing tractional detachments of the retina. In addition, panretinal photocoagulation (typically performed with a fiber optic endolaser probe intra-operatively) can be applied more effectively during pars plana vitrectomy to treat the underlying PDR. The rationale is that much of the advanced complications of DR can be treated through stimulus removal. Specifically, the fibrotic sequela and neovascular growths from the retina in towards the center of the eye (and the corresponding angiogenic stimulus and growth factors) can be mechanically “sucked out” as the vitreous body is removed through surgery leaving a angiogenic “clear zone” for a period of time. At least temporarily, PPV can positively impact the advanced DR processes. In more severe cases of DR, specifically those with tractional retinal detachment or severe nonclearing vitreous hemorrhage, vitrectomy is indicated to prevent blindness and/or severe visual loss Vitrectomy is clearly beneficial in the treatment of advanced active PDR. Well controlled studies demonstrate that early vitrectomy increases the percentage of eyes with a VA of ˜10/20 to 44%, compared with 28% in more conventionally managed patients. The use of early vitrectomy is also warranted for eyes with very severe PDR, but not for patients with less severe DR.

Given the risk of blindness without treatment, laser photocoagulation and/or vitrectomy will continue to have a major role in the management of DR/DME. Both laser photocoagulation and vitrectomy have been demonstrated to improve quality of life for patients with DR in a cost-effective fashion. However, these interventions are indicated only when DR has progressed to a measurably advanced stage in which some VA may already be lost. Side effects, such as loss of peripheral, night, or color vision have been noted by some photocoagulation-treated patients and cannot be ignored. Vitrectomy can accelerate cataract formation and includes risks of retinal detachment and endophthalmitis (a serious acute infection of the interior of the eye) but fortunately these side effects are relatively rare. It must be noted that in all cases, the eyes treated with photocoagulation may have the underlying processes driving the DR still present and the disease may continue to progress with increased need for ongoing repeat treatment.

### Pharmacological Therapies

Because of the limitations of current treatments, new pharmacological therapies are being developed, targeting the underlying biochemical mechanisms that cause DR/DME. The rationale behind the use of these agents is the prevention of diabetes-induced damage to the retinal microvasculature. The mechanisms that contribute to cellular damage in the retina include increased flux through the polyol pathway leading to sorbitol accumulation, production of advanced glycation end products (AGEs), increased oxidative stress, and activation of the protein kinase C (PKC) pathway (FIG. 1). Each of these mechanisms has been targeted with specific inhibitory compounds, some of which may become viable therapies to treat DR/DME. Blood vessel formation plays a pivotal role in the development of PDR, and various anti-angiogenic agents are also under investigation as potential therapies for DR. Because there is considerable overlap among these and other pathways in the pathogenesis of DR, combinations of therapies may prove to be more effective in preventing DR.

### SUMMARY

In accordance with a first aspect of the present disclosure there is provided a composition comprising a cannabidiol (CBD) for use in the treatment of Proliferative Diabetic Retinopathy.

Preferably the proliferative diabetic retinopathy is treatment resistant.

More preferably the proliferative diabetic retinopathy is characterized by diffuse retinal angiogenesis or focal angiogenesis with impairment.

In one embodiment the composition is used in combination with one or more concomitant antiangiogenic drugs (AAD).

In a further embodiment the CBD is present as a highly purified extract of cannabis which comprises at least 95% (w/w) CBD, more preferably 98% (w/w) CBD. Preferably, the extract comprises less than 0.15% THC. More preferably, the extract further comprises up to 1% CBDV.

In a further embodiment described herein one or more AAD is selected from the group consisting of:

Avastin (bevacicizumab), Eyelea (Aflibercept) (Regeneron), Lucentis (ranibizumab), Macugen (pegaptanib sodium), Brolucizumab (RTH-258) (Novartis), VOTRIENT® (Pazopanib), Beovu®, PAN-90806, OPT-302, ICON-1 (Iconic Therapeutics), RGX-314 (REGENXBIO), DE-122 (Carotuximab) (Santen), RG7716 (nesvacumab) (Roche), Abicipar Abicipar pegol (Allergan), KSI-301 (KODIAK Sciences), KSI-501 (KODIAK Sciences), GB-102 (Graybug vision), X-82 (Tyrogenex), AKST4290 (ALKAHEST), lBI302 (Innovent Biologics), AR-13503 (Aerie Pharmaceuticals).

Preferably, the number of different anti-angiogenic drugs that are used in combination with the CBD is reduced in comparison to standard treatment regimens for an indication. Alternatively the dose of the one or more anti-angiogenic drugs that are used in combination with the CBD is reduced in comparison to standard treatment regimens for the indication

Preferably, the dose of CBD is greater than 5 mg/kg/day.

In accordance with a second aspect of the present disclosure there is provided a method of treating proliferative diabetic retinopathy (PDR) comprising administering cannabidiol (CBI)) to a subject.

In accordance with a third aspect of the present disclosure there is provided a composition for use in the treatment of lymphangiogenesis comprising cannabidiol (CBD), a solvent, a co-solvent, a sweetener, and a flavouring.

Preferably the solvent is an edible oil, e.g., sesame oil, the co-solvent is ethanol, the sweetener is natural or artificial sweetener, e.g., sucralose, the flavouring is a natural or artificial flavouring, e.g., strawberry flavour and the CB) is present at a concentration of between 25/mg/ml and 100 mg/ml.

More preferably the composition comprises cannabidiol CBD at a concentration of between 25 to 100 mg/ml, ethanol at a concentration of 79 mg/ml, sucralose at a concentration of 0.5 mg/ml, strawberry flavoring at a concentration of 0.2 mg/ml and sesame q.s. to 1.0 ml.

### BRIEF DESCRIPTION OF FIGURES

FIG. 1 provides a schematic representation of biological targets of PKC isoform activation and synthesis.

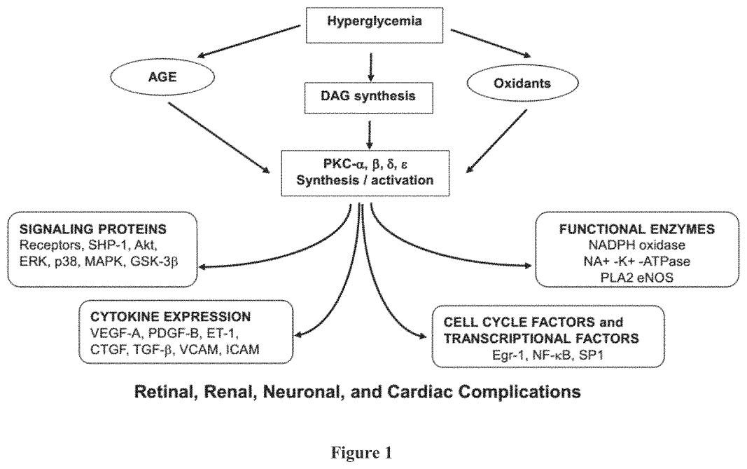

FIG. 2 provides the schematic representation of hypoglycemia-induced PKC activation affecting multiple cellular function.

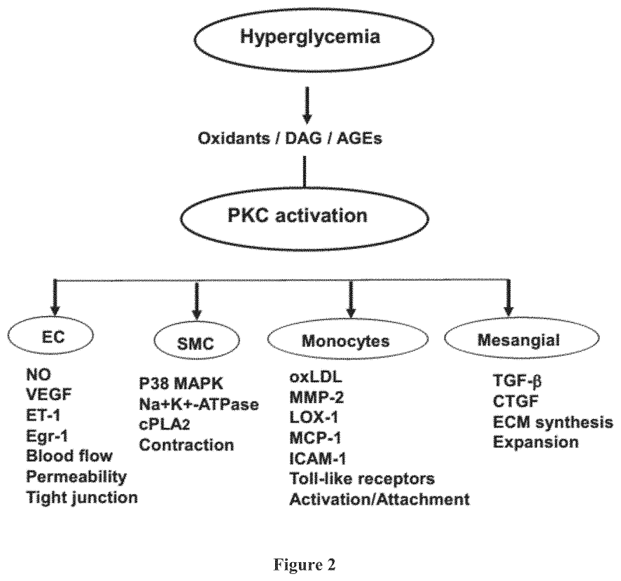

FIG. 3 illustrates ocular delivery methods of use with compositions described herein. 3A Shows ocular delivery methods including: topical administration, intravitreal injection, periocular injection, and systemic administration 3B shows delivery methods and compositions including: Ranexus® Encapsulated Technology, Ranibizumab microspheres, Ranibizumab and bevacizumab nanoparticles, Iluvien intravintravitreal implant, Ozurdex® intravintravitreal implant, Retisert® intravintravitreal implant, DSP-Visulex Cul-de-sac Implants, Surodex® Episcleral implants, drug eluting contact lenses, ocular iontophoresis, Dextenza punctum plug, and I-vation TA intravintravitreal implant.

### DETAILED DESCRIPTION

The present disclosure relates to methods and compositions for treating conditions associated with angiogenesis, and, more specifically, this disclosure relates to methods and compositions for treating conditions associated with angiogenesis using cannabidiol (CBD)-based compositions Some embodiments of the disclosure are directed to methods and compositions for treating conditions associated with lymphangiogenesis using cannabidiol (CBD). Preferably the CBD used is in the form of a highly purified extract of cannabis such that the CBD is present at greater than 98% of the total extract (w/w) and the other components of the extract are characterized.

In some embodiments, the CBD-based therapeutic composition may be present as a highly purified extract of cannabis which comprises at least 98% (w/w) CBD, less than 0.15% THC and preferably up to 1% cannabidivarin (CBDV). In some embodiments, the highly purified CBD extract comprises at least 98% (w/w) CBD, less than 0.15% THC and/or less than 0.15% delta-8 (isomeric THC) and preferably up to 1% cannabidivarin (CBDV).

In some embodiments, tetrahydrocannabinol (THC) has been substantially removed to a level of not more than 0.15% (w/w) in the composition. The delta-8 structural isomer of THC may be present in concentrations not to exceed 1.0% (w/w).

CBD may also be present as a synthetic compound. Preferably the CBD is for use in combination with any combination of dietary modification, improved glycemic control, insulin, intraocular steroids, oral hypoglycemic drugs (four classes including sulfonylureas, metformin, thiazolidinediones, and alpha-glucosidase inhibitor) and/or anti vascular endothelial growth factor (anti-VEGF) agents selected from the group consisting of insulin, triamcinolone for injection, repaglanide, natiglinide, metformin, rosiglitazone, pioglitazone, pegaptanid, ranibizumab, bevacizumab, afibercept, verteprofin, Lapatinib, Sorafenib, Sunitinib, Axitinib, Pazopanib, pan retinal photocoagulation (PRP), focal photocoagulation, and pars plana vitrectomy where the number and/or dose of antiangiogenic drugs (AAD) that is/are used in combination with the CBD is reduced.

In some embodiments, the CBD-based composition itself may act as a treatment for the inflammation that may accompany the prior listed therapeutic use compounds, laser photocoagulation, and surgical procedures such as pars plana vitrectomy.

A composition for use in the treatment of ocular angiogenesis characterized by proliferative diabetic retinopathy comprising cannabidiol (CBD), a solvent preferably sesame oil, a co-solvent preferably ethanol, a sweetener preferably sucralose and a flavoring is also provided.

In particular, tetrahydrocannabinol (TIC) has been substantially removed to a level of not more than 0.15% (w/w). Alternatively, it is a synthetically produced CBD. In use, the CBD is used either as sole treatment or concomitantly with one and/or more additional anti-angiogenic drugs/substances (AAD). Alternatively, the CBD administration separately, sequentially, or simultaneously with one or more AAD. Alternatively the combination can be provided in a single or multiple dosage form where the CBD is formulated for administration separately, sequentially, or simultaneously. It may be provided as a kit or together with instructions to administer the one and/or more components in the manner indicated.

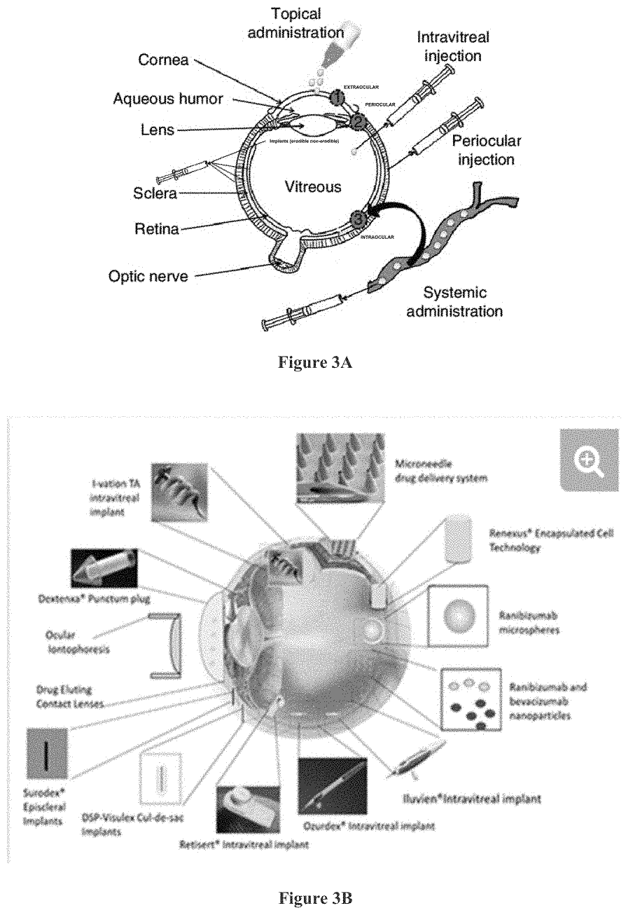

Further, administration forms may occur solely as or in combination with a liquid, gel, spray, micelles/micellar suspension, machined and/or non-machined nanoencapsulation (solution and/or suspension), liquid suspension, solid eluting form (such as within a contact lens), topical implant (upper lid, lower lid, and/or corneal ring), intra/periocular implant (biodegradable, passive elution, electric or magnetic induced release), and/or punctual eluting device (PED) (such as a plug, filament, gel, thickened suspension and/or other form meant to elute CBD (either alone or in combination with other substances) onto the surface of the eye (either via the tear film, or direct delivery via diffusion into the cornea) See, e.g., FIGS. 3A and 3B.

Existing implants, which may be used with methods and compositions described herein include, but are not limited to Ozurdex (dexamethasone), Retisert (fluocinolone acetonide), Iluvien (fluocinolone acetonide), Vitasert (gancylovir), and Sutroex (dexamethasone). Implantation may be performed using surgical insertion, insertion using applicators designed for administration of said therapeutic compositions, or insertion with needles, including 22-25 gauge needles.

Compositions of the disclosure may be delivered as implants which can have a release time of up to 1 month, up to 2 months, up to 3 months, up to 6 months, up to 1 year, up to 2 years, up to 5 years, up to 10 years, or up to 20 years.

Delivery of the CBI) based compositions may include eye drops (preserved or preservative BAK-free), injection (either intraocular, subconjunctival and/or peri-ocular), implant (either intraocular, subconjunctival and/or peri-ocular), cannula, direct irrigation (via intraocular, subconjunctival, and/or periocular means), infusion (either intraocular, subconjunctival and/or peri-ocular), electrophysiologic membrane disruption/manipulation (inducing increased intraocular penetration through transiently increased permeability), and/or manual placement, adhesion, and/or insertion.

### Definitions

Definitions of some terms used to describe the embodiments and disclosure as described herein are detailed below:

### Cannabinoids.

The cannabinoids described in the present application are listed below along with their standard abbreviations.

| | | |
|---|---|---|
| CBD | Cannabidiol | 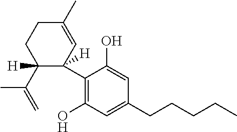 [SMILES: C=C(C)[C@@H]1CCC(C)=C[C@H]1c1c(O)cc(CCCCC)cc1O] |
| CBDA | Cannabidiolic acid | 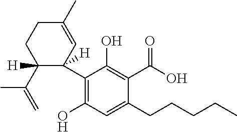 [SMILES: C=C(C)[C@@H]1CCC(C)=C[C@H]1c1c(O)cc(CCCCC)c(C(=O)O)c1O] |
| CBDV | Cannabidivarin | 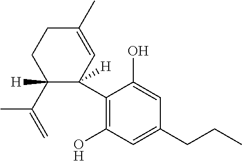 [SMILES: C=C(C)[C@@H]1CCC(C)=C[C@H]1c1c(O)cc(CCC)cc1O] |
| THC | Tetrahydrocannabinol | 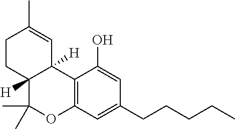 [SMILES: CCCCCc1cc(O)c2c(c1)OC(C)(C)[C@@H]1CCC(C)=C[C@@H]21] |
| | Delta-9-THC | 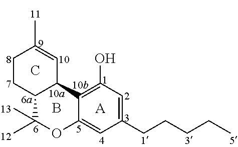 |
| | Delta-8-THC | 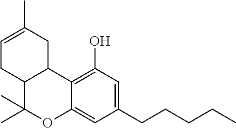 [SMILES: CCCCCc1cc(O)c2c(c1)OC(C)(C)C1CC=C(C)CC21] |

Compositions of the invention may comprise, consist of, or consist essentially of one or more cannabinoids, or S, L, or R isomers thereof.

The table above is not exhaustive and merely details the cannabinoids which are identified in the present application for reference. So far over 60 different cannabinoids have been identified and these cannabinoids can be split into different groups as follows: Phytocannabinoids; Endocannabinoids and Synthetic cannabinoids (which may be novel cannabinoids or synthetically produced phytocannabinoids or endocannabinoids).

“Phytocannabinoids” are cannabinoids that originate from nature and can be found in the cannabis plant. The phytocannabinoids can be isolated from plants to produce a highly purified extract or can be reproduced synthetically.

“Highly purified cannabinoids” are defined as cannabinoids that have been extracted from the cannabis plant and purified to the extent that other cannabinoids and non-cannabinoid components that are co-extracted with the cannabinoids have been removed, such that the highly purified cannabinoid is greater than or equal to 98% (w/w) pure.

“Synthetic cannabinoids” are compounds that have a cannabinoid or cannabinoid-like structure and are manufactured using chemical means rather than by the plant.

The term “VEGF” refers to the 165-amino acid vascular endothelial cell growth factor, and related 121-, 189-, and 206-amino acid vascular endothelial cell growth factors, as described by Leung et al., Science 246:1306 (1989), and Houck et al., Mol. Endocrin. 5:1806 (1991) together with the naturally occurring allelic and processed forms of those growth factors.

The term “VEGF receptor” or “VEGFr” refers to a cellular receptor for VEGF, ordinarily a cell-surface receptor found on vascular endothelial cells (Schematic B), as well as variants thereof which retain the ability to bind hVEGF One example of a VEGF receptor is the fins-like tyrosine kinase (flt), a transmembrane receptor in the tyrosine kinase family. DeVries et al., Science 255:989 (1992); Shibuya et al, Oncogene 5:519 (1990). The fit receptor comprises an extracellular domain, a transmembrane domain, and an intracellular domain with tyrosine kinase activity. The extracellular domain is involved in the binding of VEGF, whereas the intracellular domain is involved in signal transduction. Another example of a VEGF receptor is the flk-1 receptor (also referred to as KDR) Matthews et al., Proc Nat. Acad. Sci. 88:9026 (1991), Terman et al., Oncogene 6:1677 (1991); Terman et al., Biochem. Biophys. Res. Commun. 187:1579 (1992). Binding of VEGF to the fit receptor results in the formation of at least two high molecular weight complexes, having an apparent molecular weight of 205,000 and 300,000 Daltons. The 300,000 Dalton complex is believed to be a dimer comprising two receptor molecules bound to a single molecule of VEGF.

As used herein, a “VEGF antagonist” refers to a compound that can diminish or inhibit VEGF activity in vivo. A VEGF antagonist can bind to a VEGF receptor(s) or block VEGF protein(s) from binding to VEGF receptor(s). A VEGF antagonist can be, for example, a small molecule, an anti-VEGF antibody or antigen-binding fragments thereof, fusion protein (such as aflibercept), an aptamer, an antisense nucleic acid molecule, an interfering RNA, receptor proteins, and the like that can bind specifically to one or more VEGF proteins or one or more VEGF receptors. Several VEGF antagonists are described in WO 2006/047325.

In a preferred embodiment, the VEGF antagonist is an anti-VEGF antibody.

The term “antibody” as used herein includes whole antibodies and any antigen binding fragment (i.e., “antigen-binding portion,” “antigen binding polypeptide,” or “immunobinder”) or single chain thereof. An “antibody” includes a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen-binding portion” of an antibody (or simply “antibody portion”) refers to one or more fragments of an antibody that retain the ability to specifically bind to an antigen (e.g., VEGF). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, CL and CH1 domains; (ii) a F(ab′)₂ fragment, a bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a single domain or dAb fragment (Ward et al, (1989) Nature 341:544-546), which consists of a V_{H} domain, and (vi) an isolated complementarity determining region (CDR) or (vii) a combination of two or more isolated CDRs which may optionally be joined by a synthetic linker. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins. Antibodies can be of different isotype, for example, an IgG (e.g., an IgG1, IgG2, IgG3, or IgG4 subtype), IgA1, IgA2, IgD, IgE, or IgM antibody.

As used herein, a “mammal” includes any animal classified as a mammal, including, but not limited to, humans, domestic animals, farm animals, and companion animals, etc.

As used herein, the term “subject” or “patient” refers to human and non-human mammals, including but, not limited to, primates, rabbits, pigs, horses, dogs, cats, sheep, and cows. Preferably, a subject or patient is a human.

An “ocular disease” or “neovascular ocular disease” that can be treated using a method of the invention includes, a condition, disease, or disorder associated with ocular neovascularization, including, but not limited to, abnormal angiogenesis, choroidal neovascularization (CNV), retinal vascular permeability, retinal edema, diabetic retinopathy (particularly proliferative diabetic retinopathy), diabetic macular edema, neovascular (exudative) age-related macular degeneration (AMD), including CNV associated with nAMD (neovascular AMD), sequela associated with retinal ischemia, Central Retinal Vein Occlusion (CRVO), and posterior segment neovascularization.

Phytocannabinoids can be obtained as either the neutral (decarboxylated form) or the carboxylic acid form depending on the method used to extract the cannabinoids. For example it is known that heating the carboxylic acid form will cause most of the carboxylic acid form to decarboxylate into the neutral form.

### Preparation of Highly Purified CBD Extract

Provided herein are methods for the production of a highly-purified (>98% w/w) cannabidiol extract which has a known and constant composition. In some embodiments, said extract was used for expanded access trials described in Examples below.

In some embodiments, the drug substance is a liquid carbon dioxide extract of high-CBD containing chemotypes of Cannabis sativa L, which has been further purified by a solvent crystallization method to yield CBD. The crystallisation process specifically removes other cannabinoids and plant components to yield greater than 98% CBD.

In some embodiments, the Cannabis sativa L. plants are grown, harvested, and processed to produce a botanical extract (intermediate) and then purified by crystallization to yield the CBD (drug substance).

In some embodiments, the plant starting material is referred to as Botanical Raw Material (BRM); the botanical extract is the intermediate; and the active pharmaceutical ingredient (API) is CBD, the drug substance. Both the botanical starting material and the botanical extract are controlled by specifications. The drug substance specification is described in Table 3 below.

TABLE 3
CBD Specification

| Test | Test Method | Limits |
|---|---|---|
| Appearance | Visual | Off-white/pale yellow crystals |
| Identification A | HPLC-UV | Retention time of major peak |
| | | corresponds to certified CBD |
| | | Reference Standard |
| Identification B | GC-FID/MS | Retention time and mass spectrum |
| | | of major peak corresponds to |
| | | certified CBD Reference Standard |
| Identification C | FT-IR | Conforms to reference spectrum for |
| | | certified CBD Reference Standard |
| Identification D | Melting | 65-67° C. |
| | Point | |
| Identification E | Specific | Conforms with certified CBD |
| | Optical | Reference Standard; −110° to −140° |
| | Rotation | (in 95% ethanol) |
| Total Purity | Calculation | ≥98.0% |
| Chromatographic | HPLC-UV | ≥98.0% |

Purity 1

| | | |
|---|---|---|
| Chromatographic | GC-FID/MS | ≥98.0% |

Purity 2

| Impurities (Other | | HPLC-UV |
|---|---|---|
| Cannabinoids): | | |
| CBDA | | NMT 0.15% w/w |
| CBDV | | NMT 1.0% w/w |
| Δ⁹ THC | | NMT 0.15% w/w |
| CBD-C4 | | NMT 0.5% w/w |
| Residual Solvents: | GC | |
| Alkane | | NMT 0.5% w/w |
| Ethanol | | NMT 0.5% w/w |
| Residual Water | Karl Fischer | NMT 1.0% w/w |

NMT-Not more than

In some embodiments, the purity of the CBD is greater than 50%, greater than 60% greater than 75%, greater than 80% greater than 85%, greater than 90%, greater than 95%, or greater than 98%. In a preferred embodiment, the purity of the CBD) drug substance achieved is greater than W8%. In some embodiments, the possible impurities are related cannabinoids: CBDA, CBDV, CBD-C4 and THC.

In some embodiments, distinct chemotypes of Cannabis sativa L. plant are used to maximize the output of the specific chemical constituents, the cannabinoids One type of plant produces predominantly CBD. Only the (−)-trans isomer occurs naturally, furthermore during purification the stereochemistry of CBI) is not affected.

In some embodiments, therapeutics comprising one or more isomers selected from a group comprising D, L, R, and S isomers are used for treatment.

### Angiogenesis

The present disclosure relates, in part, to the discoveries that CBD plays a role in angiogenesis and that its modulation inhibits angiogenesis. Studies have revealed that the endocannabinoid system is involved in many biological processes including angiogenesis, pain, animal and humans. Some embodiments are directed to methods and compositions for treating conditions associated with unwanted angiogenesis, also referred to as neovascularization, using CBD. In one embodiment provides a method of treating an angiogenic condition. The method includes administering CBI) to a subject in an amount sufficient to inhibit angiogenesis.

The angiogenic condition may be, for example, cancer, diabetes, diabetic retinopathy, including proliferative diabetic retinopathy, including treatment-resistant diabetic retinopathy, age-related macular degeneration, rheumatoid arthritis, psoriasis, complications of AIDS (Kaposi's sarcoma), Alzheimer's disease, chronic inflammatory diseases (i.e. Crohn's disease and ulcerative colitis), acute inflammation, rheumatic diseases, autoimmune diseases, systemic inflammatory diseases including systemic lupus erythematosus (SLE), systemic sclerosis (SSc), Sjögren's syndrome (SS), mixed connective tissue disease (MCTD), polymyositis/dermatomyositis (PM/DM) and systemic vasculitis, endometriosis, skin diseases (i.e. psoriasis), thrombotic diseases (including diseases related to platelet function), and/or diseases related to coagulation and complement cascade. Particularly, the condition may include cancer, an ocular angiogenic condition such as unwanted choroidal neovasculature or corneal angiogenesis, scar formation, tissue repair, wound healing, atherosclerosis, and/or arthritis.

In some embodiments, the compositions as described herein are used for the treatment, prevention, or to slow progression of proliferative diabetic retinopathy is characterized by diffuse retinal angiogenesis or focal angiogenesis with impairment.

In some embodiments, the CBD-based composition(s) is used in combination with one or more concomitant antiangiogenic drugs (AAD).

Another embodiment provides a method for administering CBD to an animal or human subject in an amount sufficient to inhibit angiogenesis of the eye or ocular system. For example, the embodiments provide a method for treating unwanted choroidal neovasculature, which includes administering a CBD to a subject in an amount sufficient to inhibit the unwanted choroidal neovasculature. The subject may have age-related macular degeneration. Some embodiments are directed to methods for treating CNV subtypes such as Classic CNV, Predominantly Classic CNV, minimally classic CNV, Occult CNV without pigment epithelial detachment (PED) either including or excluding retinal angiomatous proliferation (RAP), CNV with vascularized PED (with or without RAP), and disciform scars/membranes Some embodiments also provide a method of treating corneal angiogenesis, which includes administering a CBD to a subject in an amount sufficient to inhibit the unwanted corneal angiogenesis.

Inhibition of angiogenesis (such as inhibition of unwanted tumor-related neovasculature, choroidal neovasculature, or corneal neovasculature) using the compositions and methods described herein, may include blood vessel regression and/or inhibition of blood vessel formation/function Inhibition of blood vessel formation/function may include cessation of blood vessel (both micro and macrovascularization) formation or a decrease in the rate of blood vessel growth in a treated subject as compared to an untreated subject. In some embodiments, the CBD may be administered locally and/or systemically, as a single agent treatment and/or multiple agents used concomitantly or staged as a function of time and/or response.

### Lymphangiogenesis

The present disclosure also relates, in part, to the surprising discovery that cannabinoid receptor blockage inhibits lymphangiogenesis in animal models. Accordingly, some embodiments are directed to methods and compositions for treating conditions associated with unwanted lymphangiogenesis using a cannabidiol (CBD). One aspect of the disclosure provides a method of treating a lymphangiogenic condition. The method includes administering a CBD to a subject in an amount sufficient to inhibit lymphangiogenesis. The lymphangiogenic condition may be, for example, cancer, neoplasm, metastasis, organ transplantation, particularly the organization of immunologically active lymphocytic infiltrates following organ transplantation, edema, rheumatoid arthritis, scar formation, tissue repair, psoriasis, and wound healing. Particularly, the condition may include cancer or an ocular lymphangiogenic condition such as corneal lymphangiogenesis.

In some embodiments, methods and compositions as described herein are used for the treatment or to slow progression of treatment resistant angiogenesis, including those which manifest as abnormal vascular lesions with visual impairment.

Some embodiments provide a method for treating cancer. The method includes administering a CBD to a subject in an amount sufficient to inhibit lymphangiogenesis. In certain embodiments, the lymphangiogenesis inhibition attenuates tumor growth and/or inhibits tumor metastasis.

Another aspect of the disclosure provides a method for treating an ocular lymphangiogenic condition. The method includes administering a C3D to a subject in an amount sufficient to inhibit lymphangiogenesis of the eye. One embodiment provides a method for treating corneal lymphangiogenesis, which includes administering a CBD to a subject in an amount sufficient to inhibit the unwanted corneal lymphangiogenesis.

Inhibition of lymphangiogenesis (such as inhibition of unwanted tumor-related lymph vessels or corneal lymphangiogenesis) may include lymph vessel regression and/or inhibition of lymph vessel formation. Inhibition of lymph vessel formation may include cessation of lymph vessel formation or a decrease in the rate of lymph vessel growth in a treated subject as compared to an untreated subject. In some embodiments. CBD based compositions as described herein may be administered locally or systemically. These CBDs can act as direct or indirect inhibitors of angiogenesis and/or lymphangiogenesis (Stevenson, et al., Arch Ophthalamol. 2012 January: 130(1): 90-100).

In some embodiments of the disclosure, the method may include additional treatment and/or administration of additional agents, before, during and/or after administration of the CBD. For example, photodynamic therapy treatment, administration of a VEGF inhibitor, and/or administration of an apoptosis-modulating factor, may be performed before, during, and/or after administration of one or more CBDs (Peach, et al. Int J Mol Sci. 2018 April; 19(4); 1264). The practice of this method may enhance, additively and/or synergistically, the therapeutic efficacy of the CBD and/or additional treatment and/or additional agent.

The present disclosure relates, in part, to the discoveries that CB plays a role in angiogenesis and that CB blockade inhibits angiogenesis in animal models, for example, animal models of CNV and corneal angiogenesis. Accordingly, the disclosure describes methods and compositions for treating angiogenic conditions by administering a CBD to a subject in an amount sufficient to inhibit angiogenesis. Inhibition of angiogenesis using a CBD can include blood vessel regression and/or inhibition of blood vessel formation. Inhibition of new blood vessel formation includes cessation of new blood formation and/or a decrease in the rate of new blood vessel formation, for example, as compared to an untreated control.

CB inhibition of the present embodiments may be useful in inhibiting various types of angiogenesis, for example, sprouting angiogenesis, intussusceptive angiogenesis, and/or inflammatory angiogenesis. Sprouting angiogenesis enables new vessel growth across gaps in the vasculature. It is initiated by degradation of the basement membrane supporting endothelial cells by proteases secreted from the endothelial cells. The proteases may be secreted from endothelial cells activated by mitogens, such as vascular endothelial growth factor (VEGF) and basic fibroblast growth factor (bFGF). The endothelial cells loosened from the degraded basement membrane are free to migrate and proliferate, leading to the formation of endothelial cell sprouts in the stroma. Then, vascular loops are formed and capillary tubes develop to complete the lumen of the vessel and new basement membrane is deposited. Sprouting differs from intussusceptive angiogenesis because it forms a new vessel as opposed to splitting existing vessels.

Intussusceptive or splitting angiogenesis occurs when the capillary wall grows into the lumenal space to split a single vessel in two. After the two opposing capillary walls contact one another, the endothelial cell junctions are reorganized and the vessel bilayer is perforated to allow growth factors and cells to penetrate the lumen. Then, the core is formed between the two new vessels at the zone of contact. Specifically, pericytes and myofibroblasts facilitate deposition of collagen fibers into the core to provide an extracellular matrix for growth of the vessel lumen By reorganizing existing cells in a blood vessel, intussusception allows for an increase in the number of capillaries without a corresponding increase in the number of endothelial cells. This is especially important in embryonic development as there are not enough resources to create a rich microvasculature with new cells every time a new vessel develops. Inflammatory angiogenesis occurs as a result of specific compounds inducing the creation of new blood vessels, for example new capillaries, in the body. The absence of blood vessels in a repairing or otherwise metabolically active tissue may retard repair or some other function, and inflammatory angiogenesis acts to deliver new blood vessels to such tissue. Accordingly, tumor growth and metastasis may depend on inflammatory angiogenesis.

Inflammatory angiogenesis produces blood vessels where there previously were none, which can affect the properties of the newly vascularized tissue and inhibit the proper function of the tissue. For example, the use of contact lenses may cause tissue irritation and inflammation that may lead to neovascularization. Corneal neovascularization associated with contact lens use may inhibit the proper functioning of the corneal tissue. Moreover, choroidal neovascularization of the macula that is associated with AMD may inhibit the proper functioning of the macula. Since CB is involved in the leukocyte recruitment cascade, it may be useful in inhibiting inflammatory angiogenesis, which is related to angiogenesis associated with tumor growth and metastasis, corneal neovascularization, and CNV

The present disclosure also relates, in part, to the discovery that a CB blockade inhibits lymphangiogenesis in animals, for example, animals exhibiting corneal lymphangiogenesis. Accordingly, the disclosure describes methods and compositions for treating lymphangiogenic conditions by administering a CBD to a subject in an amount sufficient to inhibit lymphangiogenesis. Inhibition of lymphangiogenesis using a CBD can include lymph vessel regression and/or inhibition of lymph vessel formation. Inhibition of new lymph vessel formation includes cessation of new lymph formation and/or a decrease in the rate of new lymph vessel formation, for example, as compared to an untreated control.

Lymphatic vessels and their formation (lymphangiogenesis) are implicated in a number of pathological conditions, such as neoplasm, metastasis, organization of immunologically active lymphocytic infiltrates following organ transplantation, edema, rheumatoid arthritis, psoriasis, and wound healing. Lymphangiogenesis has been shown to be induced by certain growth factors, by inflammation, and/or by tumor growth. Lymphangiogenesis has been shown to be induced by VEGF activation of VEGF receptor 3, and in some instances, VEGF receptor 2.

CBDs include, for example, a protein such as an antibody specific for a CB and/or the conjugate binding partner of a CB, and/or fragments thereof, as described more fully below. CBDs also include nucleic acids and small molecules as described more fully below. CB has been shown to regulate leukocyte recruitment under physiological and pathological conditions, both as an adhesion molecule and as an enzyme. Membrane-bound CB has been shown to mediate the interaction between leukocytes and activated endothelial cells in inflamed vessels.

Both the direct adhesive and enzymatic functions of CB are believed to be involved in the leukocyte recruitment cascade Previous studies have revealed that CB is identical with the cell-surface enzyme, semicarbazide-sensitive amine oxidase (SSAO), which catalyzes the deamination of primary amines, such as methylamine and aminoacetone. This reaction generates toxic formaldehyde and methylglyoxal, hydrogen peroxide and ammonia, which are known as reactive chemicals and major reactive oxygen species. Previously, SSAO activity has been detected in retinal tissues in connection with vascular permeability. Accordingly, CBDs have been investigated in connection with vascular hyperpermeable diseases and inflammatory conditions.

As noted above, the present disclosure relates, in part, to the discoveries that CB plays a role in angiogenesis and that CB blockade inhibits angiogenesis in animal models, for example, animal models of CNV and corneal angiogenesis. For example, the Examples below indicate that CB plays a role in CNV, an integral component of AMD, and in corneal angiogenesis. In the CNV model of Example 1, CB blockade significantly reduced CNV size seven days after laser-injury induction of CNV. In the corneal angiogenesis model of Example 2, the use of a CBD was shown to significantly inhibit corneal angiogenesis in animals treated with the CBD as compared to animals that did not receive the CBD [46].

Inhibition of angiogenesis includes blood vessel regression and/or inhibition of blood vessel formation. In this model, there are two ways of achieving the beneficial effects of an inhibitor First, growth of the blood vessels may be impeded. Second, new blood vessels may regress.

The present disclosure also relates, in part, to the discovery that CB blockade inhibits lymphangiogenesis in animal models, for example, animal models of corneal lymphangiogenesis. For example, in the corneal lymphangiogenesis model of Example 2, the use of a CBD was shown to inhibit corneal lymphangiogenesis in animals treated with the CBD as compared to animals that did not receive the CBD. Inhibition of lymphangiogenesis includes lymph vessel regression and/or inhibition of lymph vessel formation. For example, lymph vessels in animals treated with CBD were compared to untreated animals, following induction of lymphangiogenesis with an IL-1β pellet More lymph vessels appear in the untreated animals, indicative of new lymph vessel formation than in animals treated with a CBD. In this model, there are two ways of achieving the beneficial effects of an inhibitor. First, growth of the lymph vessels may be impeded. Second, new lymph vessels may regress.

### I. Indications of CB Inhibition

The present disclosure includes methods and compositions for treating angiogenic conditions by administering a CBD to a subject in an amount sufficient to inhibit angiogenesis. The angiogenic conditions that may treated with the methods of this disclosure include cancer, diabetes, diabetic retinopathy, age-related macular degeneration, rheumatoid arthritis, psoriasis, complications of AIDS (Kaposi's sarcoma), Alzheimer's disease, chronic inflammatory diseases (e.g. Crohn's disease and ulcerative colitis), acute inflammation, rheumatic diseases, autoimmune diseases, systemic inflammatory diseases including systemic lupus erythematosus (SLE), systemic sclerosis (SSc), Sjögren's syndrome (SS), mixed connective tissue disease (MCTD), polymyositis/dermatomyositis (PM/DM) and systemic vasculitis, endometriosis, skin diseases (e.g. psoriasis), thrombotic diseases (including diseases related to platelet function), and/or diseases related to coagulation and complement cascade Particularly, the condition may be cancer, an ocular angiogenic condition such as unwanted choroidal neovasculature or corneal angiogenesis, scar formation, tissue repair, wound healing, atherosclerosis, and/or arthritis. Moreover, the CBD can be administered to a subject in an amount sufficient to inhibit angiogenesis related to physiologic aging and/or a condition related to aging.

The present disclosure also includes methods and compositions for treating lymphangiogenic conditions by administering a CBD to a subject in an amount sufficient to inhibit lymphangiogenesis. The lymphangiogenic conditions include, for example, cancer, neoplasm, metastasis, organ transplantation, particularly the organization of immunologically active lymphocytic infiltrates following organ transplantation, edema, rheumatoid arthritis, scar formation, tissue repair, psoriasis, and wound healing. Particularly, the condition may include cancer or an ocular lymphangiogenic condition such as corneal lymphangiogenesis. Moreover, the CBD can be administered to a subject in an amount sufficient to inhibit lymphangiogenesis related to physiologic aging and/or a condition related to aging.

### CBD as a Treatment for Cancer

The disclosure provides methods for treating cancer, the second most common cause of death in Western societies. In one aspect, the methods include administering a CBD to a subject in an amount sufficient to inhibit angiogenesis. In certain embodiments, the angiogenesis inhibition attenuates tumor growth and/or inhibits tumor metastasis. In another aspect, the methods include administering a CBD to a subject in an amount sufficient to inhibit lymphangiogenesis. In certain embodiments, the lymphangiogenesis inhibition attenuates tumor growth and/or inhibits tumor metastasis.

Cancer is characterized by cells that divide in an uncontrolled fashion Most organs can be the primary source of cancer. However, the most common sites are lung, breast and prostate. Cancer cells frequently aggregate as tumors, a mass of rapidly dividing and growing cancer cells. The rapidly growing cancer cells within a tumor requires a large influx of oxygen and other essential nutrients and a means to expel waste. However, tumors often have no pre-established vessels to meet these needs.

Tumors induce vessel growth by secreting various growth factors such as VEGF and bFGF These factors induce vessel growth into the tumor, which supplies the required nutrients and expulsion of waste, and thereby allows for rapid tumor expansion. Certain cancer cells have been shown to facilitate angiogenesis by stopping the production of an anti-VEGF enzyme, PKG, which shifts the equilibrium of blood vessel growth toward angiogenesis. Angiogenesis also can facilitate cancer metastasis. Many cancers metastasize to other sites in the organism. The ensuing secondary growth of the tumor masses is then the primary health hazard in cancer patients. It is believed that cancer cells can spread within the body by different mechanisms. In order for cancer to metastasize, individual cancer cells typically leave a tumor by entering a vessel and migrating to another site within the body. Accordingly, in the absence of established vessels to the tumor, it is difficult for individual cells to migrate away from the tumor.

It has been found that some blood vessels within a tumor are comprised of a mosaic of both endothelial cells and cancerous cells, which allows for cell migration of the cancerous cells directly into the bloodstream. Alternatively, cancer may spread through the lymphatic system to distant sites in the body. Another mode of metastasis can be through direct invasion into the surrounding tissues.

Accordingly, anti-angiogenesis and anti-lymphangiogenesis factors that inhibit the vascularization of a tumor have been investigated as means for controlling cancer cell growth and metastasis. For example, anti-angiogenesis factors such as angiostatin, endostatin, tumstatin, and the anti-VEGF antibody AVASTIN® have been investigated as compounds to inhibit neovascularization of tumors. Endothelial cells are a particularly appealing target for inhibiting vessel growth to tumors because they are more stable than cancer cells, which can mutate and become resistant to treatment. However, endothelial cells growing within tumors have been shown to display genetic abnormalities, which suggests that vessels growing within tumors may also be capable of mutation and resistance. Accordingly, new mechanisms for inhibition of angiogenesis and for inhibition of lymplangiogenesis, such as treatment with a CBD, may be critical to a regimen of treatment directed at depriving a tumor of new vessel growth and/or to facilitate the regression of tumor vessels. In addition, since CB actively modulates leukocyte-endothelial cell interaction in both physiological and pathological conditions, it may be particularly useful in cancer of hematological cells and/or immune cells. There are two mechanisms by which CB inhibition may be beneficial in such conditions. First, it may inhibit release of leukemic cells from the bone marrow or other sources of origin. Second, it may inhibit recruitment of the cells in various vascular beds in the body, reducing tissue injury and leukostasis in capillaries.

It is understood that the administration of a CBD to inhibit angiogenesis as described herein can be part of a combination therapy, for example, administered with (e.g. before, during, or after) administration of any of the anti-angiogenesis factors and/or anti-lymphangiogenesis factors described above, chemotherapy treatment, and/or radiation treatment. Further, it is understood that the administration of a CBD to inhibit lymphangiogenesis as described herein can be part of a combination therapy, for example, administered with (e.g. before, during, or after) administration of any of the anti-angiogenesis factors and/or anti-lymphangiogenesis factors described above, chemotherapy treatment, and/or radiation treatment.

b. Inhibition of CB as a Treatment for Ocular Angiogenesis

Some embodiments provide an improved method for treating ocular disorders associated with unwanted ocular angiogenesis, for example, disorders associated with corneal angiogenesis and/or CNV. The method includes administering to the subject an amount of a CBD that is sufficient to inhibit angiogenesis, for example, corneal angiogenesis and/or CNV The CBD is administered in an amount sufficient to regress blood vessels or inhibit blood vessel formation in one or more regions and/or structures of the eye.

Some embodiments provide an improved method for treating ocular disorders associated with unwanted ocular lymphangiogenesis, for example, disorders associated with corneal lymphangiogenesis. The method includes administering to the subject an amount of a CBD that is sufficient to inhibit lymphangiogenesis, for example, corneal lymphangiogenesis. The CBD is administered in an amount sufficient to regress blood vessels or inhibit lymph vessel formation in one or more regions and/or structures of the eye.

Ocular angiogenesis refers to blood vessel growth within a structure of the eye, for example, the cornea or the choroid. Ocular lymphangiogenesis refers to lymph vessel growth within a structure of the eye, for example, the cornea. The cornea is the transparent front part of the eye. It is normally devoid of both blood and lymphatic vessels and, therefore, is described as being both immune privileged and angiogenic privileged. New vessel growth to the cornea is associated with a state of disease secondary to a variety of corneal insults, including contact lens use. Contact lens use commonly induces superficial new vessel growth rather than new vessel growth, for example, by deep stromal vessels. However, both superficial and serious vessel growth have been reported with use of hydrogel, polymethyl methacrylate, and rigid gas permeable contact lenses, particularly with extended wear use contact lenses.

Deep stromal new vessel growth to the cornea indicates a profound insult, for example hypoxia, and can lead to loss of optical transparency of the cornea through, for example, stromal hemorrhage, scarring, and lipid deposition. Corneal new vessel growth is believed to result from an inflammatory or hypoxic disruption, for example, by the contact lens either mechanically irritating the limbal sulcus or creating corneal hypoxia to stimulate limbal inflammation, epithelial erosion, or hypertrophy. Ocular angiogenesis and ocular lymphangiogenesis have also been observed in connection with corneal transplants.

These insults can stimulate production of angiogenic factors by local epithelial cells, keratocytes, and infiltrating leukocytes, for example, macrophages and neutrophils. Such angiogenic factors may include acidic and basic fibroblast growth factors, interleukin 1 (IL-1), and vascular endothelial growth factor (VEGF), and may stimulate a localized enzymatic degradation of the basement membrane of perilimbal vessels at the apex of a vascular loop, thereby inducing vascular endothelial cell migration and proliferation to form new blood vessels.

Choroidal angiogenesis, also referred to herein as choroidal neovascularization or CNV, is associated with conditions that include, for example, neovascular AMD, ocular histoplasmosis syndrome, pathologic myopia, angioid streaks, idiopathic disorders, choroiditis, choroidal rupture, overlying choroid nevi, and certain inflammatory diseases. Choroidal ncovascularization (CNV) is the main cause of severe vision loss in patients with age-related macular degeneration (AMD). There is evidence that inflammatory cells are critically involved in the formation of CNV lesions and play a role in the pathogenesis of age-related macular degeneration. Inflammatory cells have been found in CNV lesions that were surgically excised from AMD patients and in autopsy eyes with CNV. In particular, macrophages have been implicated in the pathogenesis of AMD due to their spatiotemporal distribution in the proximity of the CNV lesion both in humans and experimental models.

Macrophages are known to be a source of proangiogenic and inflammatory cytokines, such as vascular endothelial growth factor (VEGF) and tumor necrosis factor (TNF)-α, both of which significantly contribute to the pathogenesis of CNV. Most of the macrophages found in the proximity of the laser-induced CNV lesions following PDT likely are derived from newly recruited peripheral blood monocytes and not resident macrophages. As shown in Example 1 below, CB inhibition reduces both CNV and the presence of macrophages at the height of CNV formation in a CNV animal model.

### II. Exemplary CBDs

The term “CBD” is understood to refer to cannabidiol, one of the many cannabinoids, or chemical compounds, found in marijuana and hemp. Further, CBD refers to any molecule, for example, a protein, peptide, nucleic acid (ribonucleic acid (RNA) or deoxyribonucleic acid (DNA)), peptidyl nucleic acid, small molecule (organic compound or inorganic compound), that inhibits angiogenesis (e.g. regresses a blood vessel and/or inhibits blood vessel formation) in a subject, for example, by blocking a CBD receptor. The term “CBD” is also understood to mean any molecule, for example, a protein, peptide, nucleic acid (ribonucleic acid (RNA) or deoxyribonucleic acid (DNA)), peptidyl nucleic acid, small molecule (organic compound or inorganic compound), that inhibits lymphangiogenesis (e.g. regresses a lymph vessel and/or inhibits lymph vessel formation) in a subject. Accordingly, an “effective amount” of a CB) is an amount of a CBD sufficient to inhibit angiogenesis and/or lymphangiogenesis. A variety of CBDs may be used in the embodiments as described herein. Useful CBDs, include but are not limited to, for example, anti-CB neutralizing antibody and small molecules that bind CB to prevent or reduce its binding to its cognate receptor or ligand; peptides (for example, the peptide inhibitors, nucleic acids (for example, anti-CB aptamers; certain antibodies, antigen binding fragments thereof, and peptides that bind preferentially to CB or the CB cognate receptor or ligand; antisense nucleotides and double stranded RNA for RNAi that ultimately reduce or eliminate the production of either CB or its cognate receptor or ligand; soluble CB; and/or soluble CB cognate receptor or ligand. These CBDs can act as direct or indirect inhibitors of angiogenesis and/or lymphangiogenesis.

a. Exemplary CBDs-Protein

Antibodies (e.g., monoclonal or polyclonal antibodies) having sufficiently high binding specificity for the marker or target protein (for example, CB or its cognate receptor or ligand) can be used as CBDs. As noted above, the term “antibody” is understood to mean an intact antibody (for example, a monoclonal or polyclonal antibody); an antigen binding fragment thereof, for example, an Fv, Fab, Fab′ or (Fab′)₂ fragment; or a biosynthetic antibody binding site, for example, an sFv, as described in U.S. Pat. Nos. 5,091,513; 5,132,405, 5,258,498; and 5,482,858; and 4,704,692. A binding moiety, for example, an antibody, is understood to bind specifically to the target, for example, CB or its receptor, when the binding moiety has a binding affinity for the target greater than about 10⁵ M⁻¹, more preferably greater than about 10⁷ M⁻¹.

Antibodies against CB or its receptor may be generated using standard immunological procedures well known and described in the art. See, for example, Practical Immunology, Butt, N. R., ed., Marcel Dekker, N Y, 1984 Briefly, isolated CB or its ligand or receptor is used to raise antibodies in a xenogeneic host, such as a mouse, goat or other suitable mammal. The CB or its ligand or receptor is combined with a suitable adjuvant capable of enhancing antibody production in the host, and injected into the host, for example, by intraperitoneal administration. Any adjuvant suitable for stimulating the host's immune response may be used. A commonly used adjuvant is Freund's complete adjuvant (an emulsion comprising killed and dried microbial cells). Where multiple antigen injections are desired, the subsequent injections may comprise the antigen in combination with an incomplete adjuvant (for example, a cell-free emulsion). Polyclonal antibodies may be isolated from the antibody-producing host by extracting serum containing antibodies to the protein of interest. Monoclonal antibodies may be produced by isolating host cells that produce the desired antibody, fusing these cells with myeloma cells using standard procedures known in the immunology art, and screening for hybrid cells (hybridomas) that react specifically with the target protein and have the desired binding affinity. Antibody binding domains also may be produced biosynthetically and the amino acid sequence of the binding domain manipulated to enhance binding affinity with a preferred epitope on the target protein. Specific antibody methodologies are well understood and described in the literature. A more detailed description of their preparation can be found, for example, in Practical Immunology, Butt, W. R., ed., Marcel Dekker, New York. 1984.

Other proteins and peptides also can be used as a CBD. Proteins and peptides of the embodiments as described herein can be produced in various ways using approaches known in the art. For example, DNA molecules encoding the protein or peptide of interest are chemically synthesized, using a commercial synthesizer and known sequence information. Such synthetic DNA molecules can be ligated to other appropriate nucleotide sequences, including, e.g., expression control sequences, to produce conventional gene expression constructs encoding the desired proteins and peptides. Production of defined gene constructs is within routine skill in the art.

The nucleic acids encoding the desired proteins and peptides can be introduced (ligated) into expression vectors, which can be introduced into a host cell via standard transfection or transformation techniques known in the art. Exemplary host cells include, for example, E. coli cells. Chinese hamster ovary (CHO) cells, HeLa cells, baby hamster kidney (BHK) cells, monkey kidney cells (COS), human hepatocellular carcinoma cells (e.g., Hep G2), and myeloma cells that do not otherwise produce immunoglobulin protein. Transfected host cells can be grown under conditions that permit the host cells to express the genes of interest, for example, the genes that encode the proteins or peptides of interest. The resulting expression products can be harvested using techniques known in the art.

The particular expression and purification conditions will vary depending upon what expression system is employed. For example, if the gene is to be expressed in E. coli, it is first cloned into an expression vector. This is accomplished by positioning the engineered gene downstream from a suitable bacterial promoter, e.g., Trp or Tac, and a signal sequence, e.g., a sequence encoding fragment B of protein A (FB). The resulting expressed fusion protein typically accumulates in refractile or inclusion bodies in the cytoplasm of the cells, and may be harvested after disruption of the cells by French press or sonication. The refractile bodies then are solubilized, and the expressed proteins refolded and cleaved by the methods already established for many other recombinant proteins.

If the engineered gene is to be expressed in eukaryotic host cells, for example, myeloma cells or CHO cells, it is first inserted into an expression vector containing a suitable eukaryotic promoter, a secretion signal, and various introns. The gene construct can be transfected into myeloma cells or CHO cells using established transfection protocols. Such transfected cells can express the proteins or peptides of interest, which may be attached to a protein domain having another function Protein treatment agents, such as antibodies and exogenous proteins, are known in the art.

For example, CBDs include, but are not limited to, for example, anti-CB neutralizing antibody, peptides.

b. Exemplary CBDs-Nucleic Acids

To the extent that the CBD is a nucleic acid or peptidyl nucleic acid, such compounds may be synthesized by any of the known chemical oligonucleotide and peptidyl nucleic acid synthesis methodologies known in the art and used in antisense therapy. Anti-sense oligonucleotide and peptidyl nucleic acid sequences, usually 10 to 100 and more preferably 15 to 50 units in length, are capable of hybridizing to a gene and/or mRNA transcript and, therefore, may be used to inhibit transcription and/or translation of a target protein.

CB gene expression can be inhibited by using nucleotide sequences complementary to a regulatory region of the CB gene (e.g., the CB promoter and/or a enhancer) to form triple helical structures that prevent transcription of the CB gene in target cells. See generally. Helene (1991) Anticancer Drug Des. 6(6)-569-84, Helene et al. (1992) Ann. NY Acad. Si. 660: 27-36; and Maher (1992) Bioessays 14(12): 807-15. The antisense sequences may be modified at a base moiety, sugar moiety or phosphate backbone to improve, e.g., the stability, hybridization, or solubility of the molecule. For example, in the case of nucleotide sequences, phosphodiester linkages may be replaced by thioester linkages making the resulting molecules more resistant to nuclease degradation. Alternatively, the deoxyribose phosphate backbone of the nucleic acid molecules can be modified to generate peptide nucleic acids (see Hyrup et al. (1996) Bioorg. Med. Chem. 4(1): 5-23). Peptidyl nucleic acids have been shown to hybridize specifically to DNA and RNA under conditions of low ionic strength. Furthermore, it is appreciated that the peptidyl nucleic acid sequences, unlike regular nucleic acid sequences, are not susceptible to nuclease degradation and, therefore, are likely to have greater longevity in vivo. Furthermore, it has been found that peptidyl nucleic acid sequences bind complementary single stranded DNA and RNA strands more strongly than corresponding DNA sequences (PCT/EP92/20702). Similarly, oligoribonucleotide sequences generally are more susceptible to enzymatic attack by ribonucleases than are deoxyribonucleotide sequences, such that oligodeoxyribonucleotides are likely to have greater longevity than oligoribonucleotides for in vivo use.

Additionally, RNAi can serve as a CBD. To the extent RNAi is used, double stranded RNA (dsRNA) having one strand identical (or substantially identical) to the target mRNA (e.g. CB mRNA) sequence is introduced to a cell. The dsRNA is cleaved into small interfering RNAs (siRNAs) in the cell, and the siRNAs interact with the RNA induced silencing complex to degrade the target mRNA, ultimately destroying production of a desired protein (e.g., CB) Alternatively, the siRNA can be introduced directly. Examples of siRNAs suitable for targeting CB are described, for example, in PCT Publication No. WO 2006/134203.

Additionally, an aptamer can be used as a CBD and may target CB. Methods for identifying suitable aptamers, for example, via systemic evolution of ligands by exponential enrichment (SELEX), are known in the art and are described, for example, in Ruckman et al. (1998) J. Biol. Chem 273: 20556-20567 and Costantino et al. (1998) J. Pharm. Sci. 87: 1412-1420.

c. Exemplary CBDs—Small Molecules

To the extent that the CBD is a small molecule, either an organic or inorganic compound, such compounds may be synthesized, extracted and/or purified by standard procedures known in the art. Many small molecule CBDs are known, for example, as described in PCT Publication Nos. WO 2004/087138 (nationalized in the United States as U.S. Published Application No. 2006/0229346). WO 2004/067521, WO 2005/014530 and WO 2005/089755 and in U.S. Pat. Nos. 7,125,901 and 6,624,202. The common structural features of these known small molecule CBDs can be used to identify additional small molecules that can be used as CBDs. Accordingly, CBDs of the present embodiments include thiazole and derivatives thereof, many of which are published, for example, in PCT Publication No. WO 2004/067521 and in U.S. Published Application Nos. 2004/0236108, 2004/0259923, 2005/0096360, and 2006/0025438 and also in U.S. Pat. No. 7,125,901. CBDs of the embodiments as described herein also include hydrazine compounds and derivatives thereof, many of which are published, for example, in U.S. Pat. No. 6,624,202 and in U.S. Published Application Nos. 2002/0173521, 2002/0198189, 2003/0125360 and 2004/0106654.

For example, a CBD can have the general structure of formula (I) (hereinafter sometimes referred to as Compound (I)):

R¹—NH—X—Y—Z (I).

In formula (I), R¹ may be an acyl; X may be a bivalent residue derived from optionally substituted thiazole; Y may be a bond, lower alkylene, lower alkenylene or —CONH—; and Z may be a group of the formula:

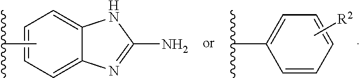

R² may be a group of the formula: -A-B-D-E wherein A may be a bond, lower alkylene, —NH— or —SO₂—; B may be a bond, lower alkylene, —CO— or —O—; D may be a bond, lower alkylene, —NH— or —CH₂NH—; and E optionally may be protected amino, —N═CH₂,

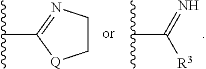

Q may be —S— or —NH—; and R³ may be hydrogen, lower alkyl, lower alkylthio or NH—R⁴ wherein R⁴ may be hydrogen, —NH₂ or lower alkyl; or a derivative thereof; or a pharmaceutically acceptable salt thereof.

In certain embodiments of formula (I), Z may be a group of the formula:

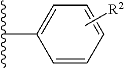

wherein R₂ may be a group of the formula:

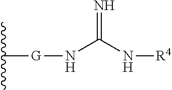

(wherein G may be a bond, —NHCOCH₂— or lower alkylene and R⁴ may be hydrogen, —NH₂ or lower alkyl); —NH₂; —CH₂NH₂; —CH₂ONH₂; —CH₂ON═CH₂;

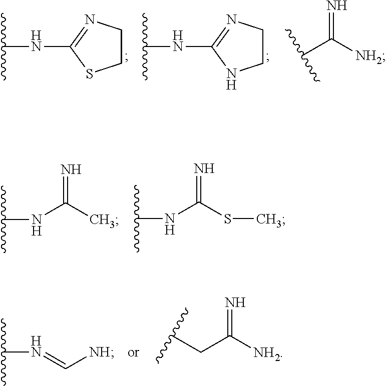

In certain embodiments of formula (I), R¹ may be alkylcarbonyl and X may be a bivalent residue derived from thiazole optionally substituted by methylsulfonylbenzyl. In certain embodiments of formula (I), X is represented by:

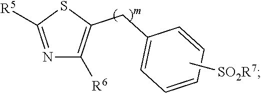

wherein, R⁵ is a bond to NH, R⁵ is a bond to Y, R⁷ is C₁-C₆, alkyl, and in is 1, 2, or 3.
Specific examples of small molecule CBDs include.
N-{4-[2-(4-{[amino (imino) methyl] amino} phenyl) ethyl]-1,3-thiazol-2-yl}acetamide;
N-[4-(2-{4-[(aminooxy)methyl]phenyl}ethyl)-1,3-thiazol-2-yl] acetamide;
N-{4-[2-(4-{[amino (imino) methyl] amino} phenyl) ethyl]-5-[4-(methylsulfonyl) benzyl]-1,3-thiazol-2-yl} acetamide;
N-{4-[2-(4-{[hydrazino (imino) methyl] amino} phenyl) ethyl]-5-[4-(methylsulfonyl) benzyl]-1,3-thiazol-2-yl}acetamide;
N-{4-[2-(4-{[hydrazino (imino) methyl] amino} phenyl) ethyl]-1,3-thiazol-2-yl}acetamide;
N-(4-{2-[4-(2-{[amino (imino) methyl] amino} ethyl) phenyl] ethyl}-1,3-thiazol-2-yl) acetamide; and derivatives thereof, or pharmaceutically acceptable salts thereof.
   Additionally, a small molecule CBD can have the structure of formula (II) (hereinafter sometimes referred to as Compound (II))

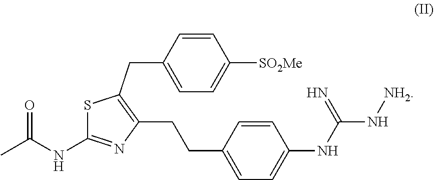 [SMILES: CC(=O)Nc1nc(CCc2ccc(NC(=N)NN)cc2)c(Cc2ccc(S(C)(=O)=O)cc2)s1]

This compound was used in Examples 1 and 2, below.

Further examples of small molecule CBDs include hydrazine compounds, as described in U.S. Pat. No. 6,624,202, having the structure of formula (III) or (IV).

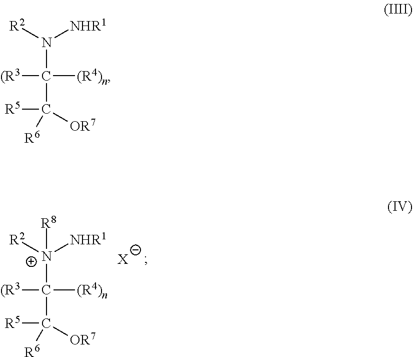

or a stereoisomer or pharmaceutically acceptable solvate, hydrate, or salt thereof.

In formula (III) or (IV) R¹ can be hydrogen, (C₁-C₄)alkyl, aralkyl, (C₂-C₅)alkanoyl, aroyl or heteroaroyl, R² can be hydrogen, or optionally substituted (C₁-C₄)alkyl, optionally substituted cycloalkyl or optionally substituted aralkyl: R³-R⁶, which can be the same or different, can be hydrogen, optionally substituted (C₁-C₄)alkyl, optionally substituted aralkyl, optionally substituted phenyl or optionally substituted heteroaryl; or R¹ and R², together with the atoms to which they are attached, can represent an optionally substituted heterocycle, or R² and R³, together with the atoms to which they are attached, can represent an optionally substituted heterocycle, or R³ and R⁵, together with the atoms to which they are attached, can represent a saturated, optionally substituted carbocycle; R⁷ can be hydrogen, (C₁-C₄)alkyl, (C₂-C₅)alkanoyl or aralkyl; R⁸ can be (C₁-C₄)alkyl or aralkyl; n can be 1, 2 or 3; and X can be chloride, bromide, iodide or R²-sulfate, where R² is as defined above with respect to formulas (III) and (IV).

### III. CB Inhibition as a Combination Therapy

CBD-based composition as described herein may be combined with other treatments for treating unwanted vasculature, such as blood vessels and/or lymphatic vessels. For example, a CBD may be administered with (e.g. before, during, or after administration of) any of the anti-angiogenesis and/or anti-lymphangiogenesis factors described herein or known in the art, chemotherapy treatment, radiation treatment, PDT therapy, treatment to modulate VEGF, gene therapy, and/or treatment to modulate apoptosis. Such combination therapy may be used to treat any condition associated with angiogenesis, including cancer and an ocular angiogenic condition such as corneal angiogenesis and unwanted CNV. Combination therapy may also be used to treat any condition associated with lymphangiogenesis, for example, cancer or an ocular lymphangiogenic condition such as corneal lymphangiogenesis.

CBD may be administered with (e.g. before, during, or after) a factor that inhibits one or more known endogenous angiogenic factors, which also may be indirectly inhibited by a CBD, including angiogenin, angiopoietin-1, Del-1, fibroblast growth factors: acidic (aFGF) and basic (bFGF), follistatin, granulocyte colony-stimulating factor (G-CSF), hepatocyte growth factor (HGF)/scatter factor (SF), interleukin-8 (IL-8), leptin, midkine, placental growth factor, platelet-derived endothelial cell growth factor (PD-ECGF), platelet-derived growth factor-BB (PDGF-BB), pleiotrophin (PTN), progranulin, proliferin, transforming growth factor-alpha (TGF-alpha), transforming growth factor-beta (TGF-beta), tumor necrosis factor-alpha (TNF-alpha), and vascular endothelial growth factor (VEGF)/vascular permeability factor (VPF).

The CBD also may be administered with one or more known endogenous angiogenesis inhibitors, including angioarrestin, angiostatin (plasminogen fragment), antiangiogenic antithrombin III, cartilage-derived inhibitor (CDI), CD59 complement fragment, endostatin (collagen XVIII fragment), fibronectin fragment, Gro-beta, heparinases, heparin hexasaccharide fragment, human chorionic gonadotropin (hCG), interferon alpha/betaigamma, interferon inducible protein (IP-10), Interleukin-12, kringle 5 (plasminogen fragment), metalloproteinase inhibitors (TIMPs), 2-methoxyestradiol, placental ribonuclease inhibitor, plasminogen activator inhibitor, platelet factor-4 (PF4), prolactin 16 kD fragment, proliferin-related protein (PRP), retinoids, tetrahydrocortisol-S, thrombospondin-1 (TSP-1), transforming growth factor-beta (TGF-b), vasculostatin, and vasostatin (calreticulin fragment).

The CBD also may be administered with one or more known chemotherapeutic agents (antineoplastic agent) including alkylating agents, antimetabolites, natural products and their derivatives, hormones and steroids (including synthetic analogs), and synthetics. Examples of compounds within these classes include alkylating agents (including nitrogen mustards, ethylenimine derivatives, alkyl sulfonates, nitrosoureas and triazenes, Uracil mustard, Chlormethine, Cyclophosphamide (Cytoxanmi), Ifosfamide, Melphalan, Chlorambucil, Pipobroman, Triethylene-melamine, Triethylenethiophosphoranine, Busulfan, Carmustine Lomustine, Streptozocin, Dacarbazine, and Temozolomide), antimetabolites (including folic acid antagonists, pyrimidine analogs, purine analogs and adenosine deaminase inhibitors, Methotrexate, 5-Fluorouracil, Floxuridine, Cytarabine, 6-Mercaptopurine, 6-Thioguanine, Fludarabine phosphate, Pentostatine, and Gemcitabine), natural products and their derivatives (including vinca alkaloids, antitumor antibiotics, enzymes, lymphokines and epipodophyllotoxins, Vinblastine, Vincristine, Vindesine, Bleomycin, Dactinomycin, Daunorubicin, Doxorubicin, Epirubicin, Idarubicin, paclitaxel (paclitaxel is commercially available as TAXOL®), Mithramycin, Deoxycoformycin, Mitomycin-C, L-Asparaginase, Interferons (especially IFN-alpha), Etoposide, and Teniposide), hormones and steroids (including synthetic analogs, 17-alpha-Ethinylestradiol, Diethylstilbestrol, Testosterone, Prednisone, Fluoxymesterone, Dromostanolone propionate, Testolactone, Megestrolacetate, Tamoxifen, Methylprednisolone, Methyltestosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, and Zoladex), and synthetics (including inorganic complexes such as platinum coordination complexes, Cisplatin, Carboplatin, Hydroxyurea, Amsacrine, Procarbazine, Mitotane, Mitoxantrone, Levamisole, and Hexamethylmelamine).

CBD-based compositions as disclosed herein can be used as adjunctive treatment for individuals who have undergone traditional therapies for PDR, therapeutic implants or gene therapy.

The CBD can be used to reduce or delay the recurrence of the condition being treated. In addition, the CBD can synergistically enhance the efficacy of the additional treatment, and/or the additional treatment may enhance the efficacy of the CBD.

In some embodiments, compositions and methods of the invention may be used to delay onset or slow progression of disease.

a. CB Inhibition in Combination with VEGF Modulation

VEGF is a known contributor to angiogenesis and to lymphangiogenesis, increasing the number of capillaries in a given network. Capillary endothelial cells have been shown to proliferate and initiate new vessel tube structures upon stimulation by VEGF Previous studies have demonstrated that plated endothelial cells presented with VEGF will proliferate, migrate, and form tube structures resembling capillaries.

VEGF has been shown to cause a massive signaling cascade in endothelial cells. Binding to VEGF receptor-2 (VEGFR-2) starts a tyrosine kinase signaling cascade that stimulates the production of factors that variously stimulate vessel permeability (eNOS, producing NO), proliferation/survival (bFGF), migration (ICAMs/VCAMs/MMPs) and finally differentiation into mature blood vessels. Moreover, as noted above, certain cancer cells stop producing an anti-VEGF enzyme, PKG, which shifts the equilibrium of blood vessel growth toward angiogenesis. Accordingly, the treatment of a CBD to inhibit angiogenesis can be combined with an anti-VEGF factor, for example, an anti-VEGF antibody or antibody fragment, nucleic acid, or small molecule. One example of an anti-VEGF factor is the anti-VEGF antibody AVASTIN®. See the URL address: gene.com/gene/products/information/oncology/avastin/index.jsp (available from Genentech, Inc., San Francisco, Calif.). Another example of an anti-VEGF factor is the aptamer MACUGEN® (see the URL address eyetk.com/science/science_vegf.asp), available from Eyetech Pharmaceuticals, Inc., NY, N.Y. Alternatively, the CBD may be combined with a VEGF specific RNAi. See the URL address: alnylam.com/therapeutic-programs/programs.asp (available from Alnylam Pharmaceuticals, Cambridge, Mass.). Similarly, the CBD may be combined with a small molecule VEGF inhibitor for the treatment of cancer, corneal neovascularization, and/or CNV.

The treatment of a CBD to inhibit lymphangiogenesis also can be combined with an anti-VEGF factor, for example, any anti-VEGF factor described above.

b. CB Inhibition in Combination with PDT

In one aspect, the disclosure provides an improved PDT-based method for treating angiogenic conditions, such as unwanted CNV and/or lymphatic conditions. An increase in efficacy and/or selectivity of the PDT, and/or reduction or delay of recurrence of the angiogenic condition, such as CNV and/or lymphatic conditions, may be achieved by administering a CBD to a subject prior to, concurrent with, or after administration of the photosensitizer. PDT involves administration of a photosensitizer to a mammal in need of such treatment in an amount sufficient to permit an effective amount (i.e., an amount sufficient to facilitate PDT) of the photosensitizer to localize in the target (e.g. the CNV). After administration of the photosensitizer, the target (e.g. the CNV) then is irradiated with laser light under conditions such that the light is absorbed by the photosensitizer. The photosensitizer, when activated by the light, generates singlet oxygen and free radicals, for example, reactive oxygen species, that result in damage to surrounding tissue. For example, PDT-induced damage of endothelial cells results in platelet adhesion and degranulation, leading to stasis and aggregation of blood cells and vascular occlusion Although this section highlights CNV, it should be understood that PDT applies to other angiogenic conditions. Moreover, this discussion also should be understood to apply to treatment of a lymphangiogenic condition.

A variety of photosensitizers that are useful in PDT include, for example, amino acid derivatives, azo dyes, xanthene derivatives, chlorins, tetrapyrrole derivatives, phthalocyanines, and assorted other photosensitizers. Amino acid derivatives include, for example, 5-aminolevulinic acid (Berg et al. (1997) J. Photochem. Photobiol. 65: 403-409; El-Far et al. (1985) Cell. Biochen. Function 3, 115-119). Azo dyes, include, for example, Sudan I, Sudan II, Sudan III. Sudan IV, Sudan Black, Disperse Orange, Disperse Red, Oil Red O, Trypan Blue, Congo Red, β-carotene (Mosky et al (1984) Exp. Res. 155, 389-396). Xanthene derivatives, include, for example, rose bengal.

Chlorins include, for example, lysyl chlorin p6 (Berg et al (1997) supra) and etiobenzochlorin (Berg et al. (1997) supra), 5, 10, 15, 20-tetra (m-hydroxyphenyl) chlorin (M-THPC). N-aspartyl chlorin e6 (Dougherty et al. (1998) J. Natl. Cancer Inst. 90: 889-905), and bacteriochlorin (Korbelik et al. (1992) J. Photochem. Photobiol. 12: 107-119). Tetrapyrrole derivatives include, for example, lutetium texaphrin (Lu-Tex, PCI-0123) (Dougherty et al. (1998) supra, Young et al. (1996) Photochem. Photobiol. 63: 892-897), benzoporphyrin derivative (BPD) (U.S. Pat. Nos. 5,171,749, 5,214,036, 5,283,255, and 5,798,349, Jori et al. (1990) Lasers Med Sci. 5, 115-120), benzoporphyrin derivative mono acid (BPD-MA) (U.S. Pat. Nos. 5,171,749, 5,214,036, 5,283,255, and 5,798,349, Berg et al. (1997) supra, Dougherty et al (1998) supra), hematoporphyrin (Hp) (Jori et al. (1990) supra), hematoporphyrin derivatives (HpD) (Berg et al. (1997) supra, West et al. (1990) In. J. Radiat. Biol. 58: 145-156), porfimer sodium or Photofrin (PIP) (Berg et al (1997) supra), Photofrin II (PII1) (He et al. (1994) Photochem. Photobiol. 59: 468473), protoporphyrin IX (PpIX) (Dougherty et al. (1998) supra, He et al. (1994) supra), meso-tetra (4-carboxyphenyl) porphine (TCPP) (Musser et al. (1982) Res. Commun. Chem. Pathol. Pharmacol. 2, 251-259), meso-tetra (4-sulfonatophenyl) porphine (TSPP) (Musser et al. (1982) supra), uroporphyrin I (UROP-I) (El-Far et al. (1985) Cell. Biochem. Function 3, 115-119), uroporphyrin III (UROP-III) (El-Far et al. (1985) supra), tin ethyl etiopurpurin (SnET2), (Dougherty et al. (1998) supra 90: 889-905) and 13,17-bis[1-carboxypropionyl] carbamoylethyl-8-etheny-2-hydroxy-3-hydroxyiminoethylidene-2,7,12,18-tetramethyl 6 porphyrin sodium (ATX-S10(Na)) Mori et al. (2000) JPN. J. Cancer Res. 91:753-759, Obana et al. (2000) Arch. Ophthalmol. 118.650-658, Obana et al. (1999) Lasers Surg. Med. 24:209-222).

Phthalocyanines include, for example, chloroaluminum phthalocyanine (AlPcCl) (Rerko et al. (1992) Photocherm. Photohiol. 55, 75-80), aluminum phthalocyanine with 2-4 sulfonate groups (AlPcS2-4) (Berg et al. (1997) supra, Glassberg et al (1991) Lasers Surg. Med. 11, 432-439), chloro-aluminum sulfonated phthalocyanine (CASPc) (Roberts et al. (1991), J. Natl. Cancer Inst. 83, 18-32), phthalocyanine (PC) (Jori et al. (1990) supra), silicon phthalocyanine (Pc4) (He et al (1998) Photochem. Photohiol. 67: 720-728, Jori et al. (1990) supra), magnesium phthalocyanine (Mg2+-PC) (Jori et al. (1990) supra), and zinc phthalocyanine (ZnPC) (Berg et al. (1997) supra). Other photosensitizers include, for example, thionin, toluidine blue, neutral red and azure c.

Useful photosensitizers also include, for example, Lutetium Texaphyrin (Lu-Tex), a new generation photosensitizer having favorable clinical properties including absorption at about 730 nm permitting deep tissue penetration and rapid clearance. Lu-Tex is available from Alcon Laboratories, Fort Worth, Tex. Other useful photosensitizers include benzoporhyrin and benzoporphyrin derivatives, for example, BPD-MA and BPD-DA, available from QLT Inc., Vancouver, Canada.

The photosensitizer preferably is formulated into a delivery system that delivers high concentrations of the photosensitizer to the CNV. Such formulations may include, for example, the combination of a photosensitizer with a carrier that delivers higher concentrations of the photosensitizer to CNV and/or coupling the photosensitizer to a specific binding ligand that binds preferentially to a specific cell surface component of the CNV.

The photosensitizer can be combined with a lipid based carrier. For example, liposomal formulations have been found to be particularly effective at delivering the photosensitizer, green porphyrin, and more particularly BPD-MA to the low-density lipoprotein component of plasma, which in turn acts as a carrier to deliver the photosensitizer more effectively to the CNV. Increased numbers of LDL receptors have been shown to be associated with CNV, and by increasing the partitioning of the photosensitizer into the lipoprotein phase of the blood, it may be delivered more efficiently to the CNV Certain photosensitizers, for example, green porphyrins, and in particular BPD-MA, interact strongly with lipoproteins. LDL itself can be used as a carrier, but LDL is more expensive and less practical than a liposomal formulation. LDL, or preferably liposomes, are thus preferred carriers for the green porphyrins since green porphyrins strongly interact with lipoproteins and are easily packaged in liposomes.

Compositions of green porphyrins formulated as lipocomplexes, including liposomes, are described, for example, in U.S. Pat. Nos. 5,214,036, 5,707,608 and 5,798,349 Liposomal formulations of green porphyrin can be obtained from QLT Inc, Vancouver, Canada. In some embodiments, other photosensitizers may likewise be formulated with lipid carriers, for example, liposomes or LIDL, to deliver the photosensitizer to CNV.

Furthermore, the photosensitizer can be coupled or conjugated to a targeting molecule that targets the photosensitizer to CNV. For example, the photosensitizer may be coupled or conjugated to a specific binding ligand that binds preferentially to a cell surface component of the CNV, for example, neovascular endothelial homing motif. It appears that a variety of cell surface ligands are expressed at higher levels in new blood vessels relative to other cells or tissues.

Endothelial cells in new blood vessels express several proteins that are absent or barely detectable in established blood vessels (Folkman (1995) Nature Medicine 1:27-31), and include integrins (Brooks et al. (1994) Science 264: 569-571; Friedlander et al. (1995) Science 270: 1500-1502) and receptors for certain angiogenic factors like VEGF. In vivo selection of phage peptide libraries have also identified peptides expressed by the vasculature that are organ-specific, implying that many tissues have vascular “addresses” (Pasqualini et al. (1996) Nature 380: 364-366). In some embodiments, a suitable targeting moiety can direct a photosensitizer to the CNV endothelium thereby increasing the efficacy and lowering the toxicity of PDT.

Several targeting molecules may be used to target photosensitizers to new vessel endothelium. For example, α-v integrins, in particular α-v β3 and α-v β5, appear to be expressed in ocular neovascular tissue, in both clinical specimens and experimental models (Corjay et al. (1997) Invest. Ophthalmol. Vis. Sci. 38, S965; Friedlander et al. (1995) supra). Accordingly, molecules that preferentially bind α-v integrins can be used to target the photosensitizer to CNV. For example, cyclic peptide antagonists of these integrins have been used to inhibit neovascularization in experimental models (Friedlander et al. (1996) Proc. Natl. Acad. Sci. USA 93:9764-9769). A peptide motif having an amino acid sequence, in an N- to C-terminal direction. ACDCRGDIXFC (SEQ ID NO: 1)—also known as RGD-4C—has been identified that selectively binds to human α-v integrins and accumulates in tumor neovasculature more effectively than other angiogenesis targeting peptides (Arap et al. (1998) Nature 279:377-380, Ellerby et al. (1999) Nature Medicine 5: 1032-1038). Angiostatin may also be used as a targeting molecule for the photosensitizer. Studies have shown, for example, that angiostatin binds specifically to ATP synthase disposed on the surface of human endothelial cells (Moser et al. (1999) Proc. Natl. Acad. Sci. USA 96:2811-2816).

Clinical and experimental evidence strongly supports a role for vascular endothelial growth factor (VEGF) in ocular new vessel growth, particularly ischemia-associated neovascularization (Adamis et al (1996) Arch. Opthamol. 114:66-71; Tolentino et al (1996) Arch. Ophthalmol. 114:964-970; Tolentino et al. (1996) Ophthalmology 103:1820-1828). Potential targeting molecules include antibodies that bind specifically to either VEGF or the VEGF receptor (VEGF-2R). Antibodies to the VEGF receptor (VEGFR-2 also known as KDR) may also bind preferentially to neovascular endothelium. VEGF receptor 3 is known to be present on lymph vessels, so a PDT method directed to lymph vessels could employ antibodies to VEGF receptor 3.

The targeting molecule may be synthesized using methodologies known and used in the art. For example, proteins and peptides may be synthesized using conventional synthetic peptide chemistries or expressed as recombinant proteins or peptides in a recombinant expression system (see, for example, “Molecular Cloning” Sambrook et al. eds, Cold Spring Harbor Laboratories). Similarly, antibodies may be prepared and purified using conventional methodologies, for example, as described in “Practical Immunology”, Butt, W. R. ed., 1984 Marcel Deckker, New York and “Antibodies, A Laboratory Approach” Harlow et al., eds. (1988), Cold Spring Harbor Press. Once created, the targeting agent may be coupled or conjugated to the photosensitizer using standard coupling chemistries, using, for example, conventional cross linking reagents, for example, heterobifunctional cross linking reagents available, for example, from Pierce, Rockford, Ill.

Once formulated, the photosensitizer may be administered in any of a wide variety of ways, for example, orally, parenterally, or rectally. Parenteral administration, such as intravenous, intralymphatic, intramuscular, or subcutaneous, is preferred Intravenous injection is especially preferred. The dose of photosensitizer can vary widely depending on the tissue to be treated; the physical delivery system in which it is carried, such as in the form of liposomes, or whether it is coupled to a target-specific ligand, such as an antibody or an immunologically active fragment.

It should be noted that the various parameters used for effective, selective photodynamic therapy in the embodiments as described herein are interrelated. Therefore, the dose should also be adjusted with respect to other parameters, for example, fluence, irradiance, duration of the light used in PDT, and time interval between administration of the dose and the therapeutic irradiation. All of these parameters should be adjusted to produce significant damage to CNV without significant damage to the surrounding tissue.

Typically, the dose of photosensitizer used is within the range of from about 0.1 to about 20 mg/kg, preferably from about 0.15 to about 5.0 mg/kg, and even more preferably from about 0.25 to about 2.0 mg/kg.

In some embodiments, the dose of CBD is greater than 5 mg/kg/day.

Furthermore, as the dosage of photosensitizer is reduced, for example, from about 2 to about 1 mg/kg in the case of green porphyrin or BPD-MA, the fluence required to close CNV may increase, for example, from about 50 to about 100 Joules/cm². Similar trends may be observed with the other photosensitizers discussed herein.

After the photosensitizer has been administered, the CNV is irradiated at a wavelength typically around the maximum absorbance of the photosensitizer, usually in the range from about 550 nm to about 750 nm. A wavelength in this range is especially preferred for enhanced penetration into bodily tissues. Preferred wavelengths used for certain photosensitizers include, for example, about 690 nm for benzoporphyrin derivative mono acid, about 630 nm for hematoporphyrin derivative, about 675 nm for chloro-aluminum sulfonated phthalocyanine, about 660 nm for tin ethyl etiopurpurin, about 730 nm for lutetium texaphyrin, about 670 nm for ATX-S10(NA), about 665 nm for N-aspartyl chlorin e6, and about 650 nm for 5, 10, 15, 20-tetra (m-hydroxyphenyl) chlorin.

As a result of being irradiated, the photosensitizer in its triplet state is thought to interact with oxygen and other compounds to form reactive intermediates, such as singlet oxygen and reactive oxygen species, which can disrupt cellular structures. Possible cellular targets include the cell membrane, mitochondria, lysosomal membranes, and the nucleus. Evidence from tumor and neovascular models indicates that occlusion of the vasculature is a major mechanism of photodynamic therapy, which occurs by damage to the endothelial cells, with subsequent platelet adhesion, degranulation, and thrombus formation.

The fluence during the irradiating treatment can vary widely, depending on the type of photosensitizer used, the type of tissue, the depth of target tissue, and the amount of overlying fluid or blood. Fluences preferably vary from about 10 to about 400 Joules/cm² and more preferably vary from about 50 to about 200 Joules/cm². The irradiance varies typically from about 50 mW/cm² to about 1800 mW/cm², more preferably from about 100 mW/cm² to about 900 mW/cm², and most preferably in the range from about 150 mW/cm² to about 600 mW/cm². In some embodiments, for many practical applications, the irradiance will be within the range of about 300 mW/cm² to about 900 mW/cm². In other embodiments, the use of higher irradiances may be selected as effective and having the advantage of shortening treatment times.

The time of light irradiation after administration of the photosensitizer may be important as one way of maximizing the selectivity of the treatment, thus minimizing damage to structures other than the target tissues. The optimum time following photosensitizer administration until light treatment can vary widely depending on the mode of administration, the form of administration such as in the form of liposomes or as a complex with LDL, and the type of target tissue. For example, bcnzoporphyrin derivative typically becomes present within the target neovasculature within one minute post administration and persists for about fifty minutes, lutetium texaphyrin typically becomes present within the target neovasculature within one minute post administration and persists for about twenty minutes, N-aspartyl chlorin e6 typically becomes present within the target neovasculature within one minute post administration and persists for about twenty minutes, and rose bengal typically becomes present in the target vasculature within one minute post administration and persists for about ten minutes. Effective vascular closure generally occurs at times in the range of about one minute to about three hours following administration of the photosensitizer. However, as with green porphyrins, it is undesirable to perform the PDT within the first five minutes following administration to prevent undue damage to retinal vessels still containing relatively high concentrations of photosensitizer.

The efficacy of PDT may be monitored using conventional methodologies, for example, via fundus photography or angiography. Closure can usually be observed angiographically by hypofluorescence in the treated areas in the early angiographic frames. During the later angiographic frames, a corona of hyperfluorescence may begin to appear which then fills the treated area, possibly representing leakage from the adjacent choriocapillaris through damaged retinal pigment epithelium in the treated area. Large retinal vessels in the treated area typically perfuse following photodynamic therapy. Minimal retinal damage is generally found on histopathologic correlation and is dependent on the fluence and the time interval after irradiation that the photosensitizer is administered. In some embodiments, the choice of appropriate photosensitizer, dosage, mode of administration, formulation, timing post administration prior to irradiation, and irradiation parameters may be determined empirically. The administration of a CBD may be used before, during, and/or after PDT treatment to enhance the success of inhibiting angiogenic conditions, such as CNV, and/or lymphatic conditions.

c. CB Inhibition in Combination with an Apoptosis Factor

The efficacy of CB inhibition of angiogenesis, alone or in combination with another therapy, for example PDT, may be enhanced by combination with administration of an apoptosis-modulating factor. Similarly, the efficacy of CB inhibition of lymphangiogenesis, alone or in combination with another therapy, may be enhanced by combination with administration of an apoptosis-modulating factor. An apoptosis-modulating factor can be any factor, for example, a protein (for example a growth factor or antibody), peptide, nucleic acid (for example, an antisense oligonucleotide or siRNA), peptidyl nucleic acid (for example, an antisense molecule), organic molecule or inorganic molecule, that induces or represses apoptosis in a particular cell type. For example, it may be advantageous to prime the apoptotic machinery of endothelial cells (e.g CNV endothelial cells) with an inducer of apoptosis prior to treatment so as to increase their sensitivity to treatment. Endothelial cells primed in this manner are more susceptible to treatments such as PDT This approach may also reduce the light dose (fluence) required to achieve CNV closure in PDT and thereby decrease the level of damage on surrounding cells such as RPE. Alternatively, the cells outside the CNV may be primed with a repressor of apoptosis so as to decrease their sensitivity to the treatment. Although this section highlights CNV, it should be understood that apoptosis modulators can be used in combination with CBDs to treat other angiogenic conditions and/or lymphangiogenic conditions. Apoptosis involves the activation of a genetically determined cell suicide program that results in a morphologically distinct form of cell death characterized by cell shrinkage, nuclear condensation, DNA fragmentation, membrane reorganization and blebbing (Kerr et al. (1972) Br. J. Cancer 26: 239-257). At the core of this process lies a conserved set of proenzymes, called caspases, and two important members of this family are caspases 3 and 7 (Nicholson et al. (1997) TIBS 22:299-306). Monitoring their activity can be used to assess on-going apoptosis.

It has been suggested that apoptosis is associated with the generation of reactive oxygen species, and that the product of the Bcl-2 gene protects cells against apoptosis by inhibiting the generation or the action of the reactive oxygen species (Hockenbery et al. (1993) Cell 75: 241-251, Kane et al. (1993) Science 262: 1274-1277, Veis et al. (1993) Cell 75: 229-240, Virgili et al. (1998) Free Radicals Biol. Med. 24: 93-101). Bcl-2 belongs to a growing family of apoptosis regulatory gene products, which may either be death antagonists (Bcl-2, Bcl-xL) or death agonists (Bax, Bak) (Kroemer et al. (1997) Nat. Med. 3: 614-620). Control of cell death appears to be regulated by these interactions and by constitutive activities of the various family members (Hockenbery et al. (1993) Cell 75: 241-251) Several apoptotic pathways may coexist in mammalian cells that are preferentially activated in a stimulus-, stage-, context-specific and cell-type manner (Hakem et al. (1998) Cell 94: 339-352).

The apoptosis-inducing factor preferably is a protein or peptide capable of inducing apoptosis in cells, for example, endothelial cells, disposed in the CNV. One apoptosis inducing peptide comprises an amino sequence having, in an N- to C-terminal direction, KLAKLAKKLAKLAK (SEQ ID NO: 2). This peptide reportedly is non-toxic outside cells, but becomes toxic when internalized into targeted cells by disrupting mitochondrial membranes (Ellerby et al. (1999) supra). This sequence may be coupled, either by means of a cross-linking agent or a peptide bond, to a targeting domain, for example, the amino acid sequence known as RGD-4C (Ellerby et al. (1999) supra) that reportedly can direct the apoptosis-inducing peptide to endothelial cells. Other apoptosis-inducing factors include, for example, constatin (Kamphaus et al. (2000)J. Biol. Chem. 14: 1209-1215), tissue necrosis factor α (Lucas et al. (1998) Blood 92: 4730-4741) including bioactive fragments and analogs thereof, cycloheximide (O'Connor et al. (2000) Am. J. Pathol. 156: 393-398), tunicamycin (Martinez et al. (2000) Adv. Exp. Med. Biol. 476: 197-208), and adenosine (Harrington et al. (2000) Am. J. Physiol. Lung Cell Mol. Physiol. 279: 733-742). Furthermore, other apoptosis-inducing factors may include, for example, anti-sense nucleic acid or peptidyl nucleic acid sequences that reduce or turn off the expression of one or more of the death antagonists, for example (Bcl-2, Bcl-xL). Antisense nucleotides directed against Bcl-2 have been shown to reduce the expression of Bcl-2 protein in certain lines together with increased phototoxicity and susceptibility to apoptosis during PDT (Zhang et al. (1999) Photochem. Photobiol. 69: 582-586). Furthermore, an 18mer phosphorothiate oligonucleotide complementary to the first six codons of the Bcl-2 open reading frame, and known as G3139, is being tested in humans as a treatment for non-Hodgkins' lymphoma.

Apoptosis-repressing factors include, survivin, including bioactive fragments and analogs thereof (Papapetropoulos et al (2000) J. Biol. Chem. 275: 9102-9105), CD39 (Goepfert et al. (2000) Mol. Med. 6: 591-603), BDNF (Caffe et al. (2001) Invest. Ophthalmol. Vis. Sci. 42: 275-82), FGF2 (Bryckaert et al (1999) Oncogene 18: 7584-7593), Caspase inhibitors (Ekert et al. (1999) Cell Death Differ 6: 1081-1068) and pigment epithelium-derived growth factor including bioactive fragments and analogs thereof. Furthermore, other apoptosis-repressing factors may include, for example, anti-sense nucleic acid or peptidyl nucleic acid sequences that reduce or turn off the expression of one or more of the death agonists, for example (Bax, Bak). To the extent that the apoptosis-modulating factor is a protein or peptide, nucleic acid, peptidyl nucleic acid, or organic or inorganic compound, it may be synthesized and purified by one or more the methodologies described relating to the synthesis of the CBD above.

The type and amount of apoptosis-modulating factor to be administered may depend upon the treatment and cell type to be treated. In some embodiments, optimal apoptosis-modulating factors, modes of administration and dosages may be determined empirically. The apoptosis modulating factor may be administered in a pharmaceutically acceptable carrier or vehicle so that administration does not otherwise adversely affect the recipient's electrolyte and/or volume balance. The carrier may comprise, for example, physiologic saline.

Protein, peptide or nucleic acid based apoptosis modulators can be administered at doses ranging, for example, from about 0.001 to about 500 mg/kg, more preferably from about 0.01 to about 250 mg/kg, and most preferably from about 0.1 to about 100 mg/kg. For example, nucleic acid-based apoptosis inducers, for example, G318, may be administered at doses ranging from about 1 to about 20 mg/kg daily. Furthermore, antibodies may be administered intravenously at doses ranging from about 0.1 to about 5 mg/kg once every two to four weeks. With regard to intravitreal administration, the apoptosis modulators, for example, antibodies, may be administered periodically as bolus dosages ranging from about 10 pg to about 5 mg/eye and more preferably from about 100 μg to about 2 mg/eye.

The apoptosis-modulating factor can be administered before, during or after CBD administration. To the extent the apoptosis-modulating factor is used with PDT, it preferably is administered to the mammal prior to PDT (although it may be administered during or after PDT). Accordingly, it is preferable to administer the apoptosis-modulating factor prior to administration of the photosensitizer. The apoptosis-modulating factor, like the photosensitizer and CBD, may be administered in any one of a wide variety of ways, for example, orally, parenterally, or rectally. However, parenteral administration, such as intravenous, intramuscular, subcutaneous, and intravitreal is preferred. Administration may be provided as a periodic bolus (for example, intravenously or intravitreally) or by continuous infusion from an internal reservoir (for example, bioerodable implant disposed at an intra- or extra-ocular location) or an external reservoir (for example, and intravenous bag). The apoptosis modulating factor may be administered locally, for example, by continuous release from a sustained release drug delivery device immobilized to an inner wall of the eye or via targeted trans-scleral controlled release into the choroid (see, PCT/US00/00207).

### IV. CBD Administration and Dosing

The type and amount of CBD to be administered will depend upon the particular treatment and cell type to be treated. Optimal CBDs, modes of administration and dosages may be determined empirically. The CBD may be administered in a pharmaceutically acceptable carrier or vehicle so that administration does not otherwise adversely affect the recipient's electrolyte and/or volume balance.

Small molecule CBDs may be administered at doses ranging, for example, from 1-1500 mg/m², for example, about 3, 30, 60, 90, 180, 300, 600, 900, 1200 or 1500 mg/m². The daily dose of a composition comprising one or more small molecule CBDs as the active ingredient is, for example, up to 1 mg, 10 mg, 100 mg, 1000 mg or 2500 mg CBD/day. Protein, peptide or nucleic acid based CBDs can be administered at doses ranging, for example, from about 0.001 to about 500 mg/kg, more preferably from about 0.01 to about 250 mg/kg, and most preferably from about 0.1 to about 100 mg/kg. The CBD may be administered in any one of a wide variety of routes, for example, by a topical, transdermal, intraperitoneal, intracranial, intracerebroventricular, intracerebral, intravaginal, intrauterine, oral, rectal, parenteral (e.g., intravenous, intralymphatic, intraspinal, subcutaneous or intramuscular), and intravitreal route. With regard to intravitreal administration, the CBD, for example, anti-CB neutralizing antibody, may be administered periodically as boluses at dosages ranging from about 10 gg to about 5 mg/eye and more preferably from about 100 μg to about 2 mg/eye.

Formulations suitable for administration of a CBD may include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) condition requiring only the addition of the sterile liquid carrier, for example, water for injections, immediately prior to use.

The formulations may also be presented in continuous release vehicles. Extemporaneous injection solutions and suspensions may be prepared from sterile powders, granules and tablets of the kind previously described. The excipient formulations conveniently may be prepared by conventional pharmaceutical techniques. Such techniques include the step of bringing into association the active ingredient and the pharmaceutical carrier(s) or excipient(s). In general, the formulations are prepared by uniformly and intimately bringing into association the active ingredient with liquid carriers or finely divided solid carriers or both, and then, if necessary, shaping the product.

The CBD may be administered in a single bolus, in multiple boluses, or in a continuous release format. Accordingly, formulations may contain a single dose or unit, multiple doses or units, or a dosage for extended delivery of the CBD It should be understood that in addition to the ingredients mentioned above, the formulations of the present embodiments may include other agents conventional in the art having regard to the type of delivery in question. For example, the carrier may comprise, for example, physiologic saline, or may comprise components necessary for, for example, administration as an ointment, administration via encapsulated microspheres or liposomes, or administration via a device for continuous release. The CBD may be administered 1×, 2×, 3×, 4× or 5× daily.

The CBD also may be administered systemically or locally. For example, administration may be provided locally as a single bolus, for example, by parenteral or intravitreal injection or by deposition to a site of interest such as a location in the eye or adjacent to or within a tumor Administration may be provided systemically as a periodic bolus, for example, intravenously, intralymphatically, or intravitreally, or locally as a periodic bolus, for example, by injection, deposition, or as periodic infusion from an internal reservoir or from an external reservoir (for example, from an intravenous bag). The CBD may be administered systemically or locally in a continuous release format, for example, from a bioerodable implant or from a sustained release drug delivery device. For example, in certain embodiments, a delivery device can be used for delivery of the CBD into the eye or via targeted trans-scleral controlled release (see, PCT/US00/00207) for treatment of the eye. In certain embodiments, particularly those directed to treatment of ocular diseases, such as corneal angiogenesis, the CBD may be administered from a contact lens. The contact lens may be pre-soaked with the CBD prior to use of the contact lens. Alternatively, in certain embodiments, particularly those directed to treatment of tumors, the CBD may be incorporated into a biodegradable polymer that may be implanted at the site of a tumor. Alternatively, a biodegradable polymer may be implanted so that the CBD is slowly released systemically rather than locally. Such biodegradable polymers and their use are known in the art and described, for example, in detail in Brem et al. (1991), J. Neurosurg. 74:441-446. Osmotic minipumps may also be used to provide controlled delivery of high concentrations of CBD through cannulae to the site of interest, such as directly into a metastatic growth or into the vascular or lymphatic supply of a tumor, or to a location in the body that facilitates systemic release.

The present embodiments, therefore, include the use of a CBD in the preparation of a medicament for treating a condition associated with angiogenesis, for example, cancer, ocular angiogenesis, corneal neovascularization, and/or CNV Some embodiments also include the use of a CBD in the preparation of a medicament for treating a condition associated with lymphangiogenesis, for example, cancer, ocular lymphangiogenesis, and lymphangiogenesis of the cornea. The CBD may be provided in a kit which optionally may comprise a package insert with instructions for how to treat such a condition.

In combination treatments, the CBD may be administered to the subject prior to other treatment(s). It may alternatively or additionally be administered during and/or after the other treatment(s). In combination with PDT therapy, the CBD may be administered before, during, or after PDT therapy it may be preferable to administer the CBD prior to administration of the photosensitizer. For a combination product with PDT, a composition may provide both a photosensitizer and a CBD. The composition may also comprise a pharmaceutically acceptable carrier or excipient Thus, the present disclosure includes a pharmaceutically acceptable composition comprising a photosensitizer and a CBD; as well as the composition for use in medicine. However, the CBD and a photosensitizer may be administered separately. Instructions for such administration may be provided with the CBD and/or with the photosensitizer. If desired, the CBD and photosensitizer may be provided together in a kit, optionally including a package insert with instructions for use. The CBD and photosensitizer preferably are provided in separate containers.

The composition comprising CBD may be used in combination with other compositions and procedures for the treatment of a cancer. For example, a tumor may be treated conventionally with surgery, radiation or chemotherapy combined with the CBI) Optionally, the CBD may also be subsequently administered to the patient to extend the dormancy of metastases and to stabilize any residual primary tumor. Administration of therapeutics directed to cancer treatment are known in the art. For example, radiation therapy, including x-rays or gamma rays, are delivered from either an externally applied beam or by implantation of tiny radioactive sources. Administration of chemotherapeutic agents are well known and described in standard literature, for example, “Physicians' Desk Reference” (PDR), e.g., 2004 edition (Thomson PDR, Montvale, N.J. 07645-1742, USA). A CB) may be administered in combination with any known anti-cancer treatment and may have dosage ranges described herein. In some embodiments compositions described herein are used sequentially with known pharmaceutically acceptable agent(s) when a multiple combination formulation is inappropriate.

The foregoing methods and compositions of the disclosure are useful in treating angiogenesis and thereby ameliorate the symptoms of various disorders associated with angiogenesis including, for example, cancer (e.g. tumor growth or metastasis), corneal neovascularization, unwanted choroidal neovasculature, and AMD. The foregoing methods and compositions of the disclosure are also useful in treating lymphangiogenesis and thereby ameliorate the symptoms of various disorders associated with lymphangiogenesis including, for example, cancer (e.g. tumor growth or metastasis) and growth of lymph vessels into the cornea.

Methods and compositions as described herein are also be useful in treating other forms of angiogenesis and/or lymphangiogenesis, as described above Some embodiments of the disclosure are illustrated further by reference to the following non-limiting examples.

### Preparation of Highly Purified CBD Extract

The following describes the production of the highly-purified (>98% w/w) cannabidiol extract which has a known and constant composition which was used for the expanded access trials described in Examples below.

In summary the drug substance used in the trials is a liquid carbon dioxide extract of high-CBD containing chemotypes of Cannabis saliva L, which had been further purified by a solvent crystallization method to yield CBD. The crystallisation process specifically removes other cannabinoids and plant components to yield greater than 98% CBD

The Cannabis sativa L. plants are grown, harvested, and processed to produce a botanical extract (intermediate) and then purified by crystallization to yield the CBI) (drug substance).

The plant starting material is referred to as Botanical Raw Material (BRM); the botanical extract is the intermediate; and the active pharmaceutical ingredient (API) is CBD, the drug substance.

Both the botanical starting material and the botanical extract are controlled by specifications. The drug substance specification is described in Table 4 below.

TABLE 4
CBD Specification

| Test | Test Method | Limits |
|---|---|---|
| Appearance | Visual | Off-white/pale yellow crystals |
| Identification A | HPLC-UV | Retention time of major peak |
| | | corresponds to certified CBD |
| | | Reference Standard |
| Identification B | GC-FID/MS | Retention time and mass spectrum |
| | | of major peak corresponds to |
| | | certified CBD Reference Standard |
| Identification C | FT-IR | Conforms to reference spectrum for |
| | | certified CBD Reference Standard. |
| Identification D | Melting | 6567° C. |
| | Point | |
| Identification E | Specific | Conforms with certified CBD |
| | Optical | Reference Standard; −110° to −140° |
| | Rotation | (in 95% ethanol) |
| Total Purity | Calculation | ≥98.0% |
| Chromatographic | HPLC-UV | ≥98.0% |

Purity 1

| | | |
|---|---|---|
| Chromatographic | GC-FID/MS | ≥98.0% |

Purity 2

| Impurities (Other | HPLC-UV | |
|---|---|---|
| Cannabinoids): | | |
| CBDA | | NMT 0.15% w/w |
| CBDV | | NMT 1.0% w/w |
| Δ⁹ THC | | NMT 0.15% w/w |
| CBD-C4 | | NMT 0.5% w/w |
| Residual Solvents: | GC | |
| Alkane | | NMT 0.5% w/w |
| Ethanol | | NMT 0.5% w/w |
| Residual Water | Karl Fischer | NMT 1.0% w/w |

NMT-Not more than
1. The purity of the CBD drug substance achieved is greater than 98%. The possible impurities are related cannabinoids: CBDA, CBDV, CBD-C4 and THC.

Distinct chemotypes of Cannabis sativa L. plant have been produced to maximize the output of the specific chemical constituents, the cannabinoids. One type of plant produces predominantly CBD. Only the (−)-trans isomer occurs naturally, furthermore during purification the stereochemistry of CBD is not affected.

### Production of the Intermediate

An overview of the steps to produce a botanical extract, the intermediate, are as follows.
1. Growing
2. Decarboxylation
3 Extraction No. I-using liquid C02
4. Extraction No. 2-‘winterization’ using ethanol
5. Filtration
6. Evaporation

### EXAMPLES

### Example 1: Extraction and Purification of CBD

a. Production of CBD Extract
High CBD chemovars were grown, harvested and dried and stored in a dry room until required. The botanical raw material (BRM) was finely chopped using an Apex mill fitted with a 1 mm screen. The milled BRM was stored in a freezer for up to 3 months prior to extraction. Decarboxylation of CBDA to CBD was carried out using a large Heraeus tray oven. The decarboxylation batch size in the Heraeus is approximately 15 Kg. Trays were placed in the oven and heated to 105° C., the BRM took 96.25 minutes to reach 105° C. Held at 105° C. for 15 Minutes. Oven then set to 150° C.; the BRM took 75.7 minutes to reach 150° C.; BRM held at 150° C. for 130 Minutes. Total time in the oven was 380 Minutes, including 45 minutes cooling and 15 Minutes venting.
Extraction No. 1 was performed using liquid CO2 at 60 bar/10° C. to produce botanical drug substance (BDS) which was used for crystallization to produce the test material.
The crude CBD BDS was winterized in Extraction No 2 under standard conditions (2 volumes of ethanol at minus 20° C. for around 50 hours) The precipitated waxes were removed by filtration and the solvent evaporated using the rotary evaporator (water bath up to 60° C.) to yield the BDS.
b. Purification of the Drug Substance
The manufacturing steps to produce the drug substance from the intermediate botanical extract are as follows:
1. Crystallization using C5-C12 straight chain or branched alkane

### 2. Filtration

3. Optional recrystallization from C5-C12 straight chain or branched alkane
4. Vacuum drying
Intermediate botanical extract (12 kg) produced using the methodology above was dispersed in C5-C12 straight chain or branched alkane (9000 ml, 0.75 vols) in a 30 liter stainless steel vessel. The mixture was manually agitated to break up any lumps and the sealed container then placed in a freezer for approximately 48 hours.
The crystals were isolated by vacuum filtration, washed with aliquots of cold C5-C12 straight chain or branched alkane (total 12000 ml), and dried under a vacuum of <10 mb at a temperature of 60° C. until dry before submitting the drug substance for analysis.
The dried product was stored in a freezer at minus 20° C. in a pharmaceutical grade stainless steel container, with FDA food grade approved silicone seal and clamps.

### Example 2: CB Blockade Suppresses CNV

CB is an endothelial cell adhesion molecule involved in leukocyte recruitment. Macrophages play an important role in the development of choroidal neovascularization (CNV), an integral component of age-related macular degeneration (AMD). Previously, it was shown that CB is involved in ocular inflammation. In this Example, the expression of CB in the choroid and its role in CNV development was investigated.

These data show that CB was expressed in the choroid, exclusively in the vessels, and co-localized in the vessels of the CNV lesions. In addition, these data show that CB blockade with a specific inhibitor (Compound II, described above) significantly decreased CNV size, fluorescent angiographic leakage, and the accumulation of macrophages in the CNV lesions. Further, these data show that CB blockade significantly reduced the expression of inflammation-associated molecules such as tumor necrosis factor (TNF-α), monocyte chemoattractant protein (MCP-1) and intercellular adhesion molecule (ICAM-1). Overall, these data provide evidence for an important role of CB in the recruitment ofmacrophages to CNV lesions and identifies CB inhibition as a therapeutic strategy in the treatment of CNV.

a. Background

Choroidal neovascularization (CNV) is the main cause of severe vision loss in patients with age-related macular degeneration (AMD). There is evidence that inflammatory cells are critically involved in the formation of CNV lesions and play a role in the pathogenesis of age-related macular degeneration. Inflammatory cells have been found in the CNV lesions that were surgically excised from AMD patients and in autopsy eyes with CNV In particular, macrophages have been implicated in the pathogenesis of AMD due to their spatiotemporal distribution in the proximity of the CNV lesion both in humans and experimental models.] Macrophages are known to be a source of proangiogenic and inflammatory cytokines, such as vascular endothelial growth factor (VEGF) and tumor necrosis factor (TNF)-α, both of which significantly contribute to the pathogenesis of CNV. Most of the macrophages found in the proximity of the laser-induced CNV lesions likely are derived from newly recruited peripheral blood monocytes and not resident macrophages. Since macrophages play such a critical role in CNV formation, prevention of monocyte recruitment and infiltration into ocular tissues may ameliorate the development of CNV.

CB is an endothelial cell adhesion molecule involved in leukocyte recruitment. In ocular tissues. CB has been shown to localize on the endothelial cells of the retina and play a critical role in the recruitment of leukocytes under both normal and inflammatory conditions. Recently, it has been reported that CB antibody treatment suppresses recruitment of monocyte, macrophage lineages in vivo, suggesting an important role for CB in macrophage transmigration under pathologic conditions.

Therefore, these investigations were carried out to show that CB regulates macrophage recruitment into ocular tissues and that its blockade attenuates CNV formation. Specifically, these investigations identified the expression and distribution of CB in the choroidal tissues of normal and laser-injured animals, and investigated the role of CB in CNV formation using a specific inhibitor identified as Compound II, above.

### Methods

b. Experimental Animals

For reverse transcription polymerase chain reaction (RT-PCR) detection and immunofluorescence staining of CB in the choroid, Lewis rats (8-10 weeks old, Charles River Laboratories, Inc., Wilmington, Mass.) were used. To generate CNV in the laser injury model, Brown-Norway rats (10-12 weeks old, Charles River Laboratories, Inc., Wilmington, Mass.) were used. Rats were housed in plastic cages in a climate controlled animal facility and were fed laboratory chow and water ad libitum. All animal experiments were conducted in accordance with the ARVO Statement for the Use of Animals in Ophthalmic and Vision Research.

c. RNA Extraction and RT-PCR

Lewis rats were euthanized by overdose anesthesia and perfused with PBS (500 ml/kg body weight (BW)). Eyes were immediately enucleated and the retinal pigment epithelium (RPE)-choroid complex was obtained from the rat eyes and homogenized in extraction reagent (TRIzol Reagent; Invitrogen, Carlsbad, Calif.). As a control, the retinal tissues were separately obtained and processed. Total RNA was prepared according to the manufacturer's protocol, and equal amounts (1 pg) of total RNA were reverse transcribed with a First-Strand cDNA synthesis kit (GE Healthcare, Buckinghamshire, UK) at 37° C. for 1 hour in a 15 μl reaction volume. PCR was performed using Platinum PCR SuperMix (Invitrogen) with a thermal controller (GeneAmp PCR System 9700: Applied Biosystems, Foster city, CA). The thermal cycle was 1 minute at 94° C., 1 minute at 55° C. and 1 minute at 72° C., followed by 5 minutes at 72° C. The reaction was performed for 35 cycles for amplification of CB and 30 cycles for glyceraldehyde-3-phosphate dehydrogenase (GAPDH) with previously designed primers. The nucleotide sequences of the PCR primers were 5′-GAC CCT CGG ACA ACT GTG TCT T-3′(forward) (SEQ ID NO: 3) and 5′-GCG TT GTA GAA GCA ACA GTG A-3′ (reverse) (SEQ ID NO 4) for CB and 5′-TGG CAC AGT CAA GGC TGA GA-3′ (forward) (SEQ ID NO: 5) and 5′-CUT CTG AGT GGC AGT GAT GG-3′ (reverse) (SEQ ID NO 6) for glyceraldehyde-3-phosphate dehydrogenase (GAPDH). PCR products were analyzed by electrophoresis in a 1.5% agarose gel and stained with ethidium bromide (0.2 pg/ml). The expected sizes of the amplified cDNA fragments of CB and GAPDH were 341 bp and 387 bp, respectively Band densities were quantified using NIH Image 1.41 software (available by ftp from zippy.nimh.nih.gov/or from the web site, rsb info.nih.gov/nih-image: developed by Wayne Rasband, National Institutes of Health, Bethesda, Md.). The expression level of CB mRNA was normalized by that of GAPDH. Brown-Norway rats were anesthetized with 0.2-0.3 ml of a 50:50 mixture of 100 mg/ml Ketamine and 20 mg/ml Xylazine. Pupils were dilated with 5.0% Phenylephrine and 0.8% Tropicamide. CNV was induced with a 532 nm laser (Oculight GLx, Iridex, Mountain View, Calif.). Six laser spots (150 mW, 100 μm, 100 msec) were placed in each eye using a slit-lamp delivery system and a cover glass as a contact lens. Production of a bubble at the time of laser confirmed the rupture of the Bruch's membrane.

d. Immunohistochemistry

Seven days after laser injury paraffin sections of the choroidal-scleral complex and OCT compound-embedded sections of the rat eyes were prepared. The sections were incubated with blocking solution (Invitrogen) and then reacted with either mouse monoclonal antibody against rat CB (1:200; BD biosciences, Franklin Lakes, N.J.) or rabbit polyclonal antibody against rat CB (1.200, Santa Cruz Biotechnology, Inc) For the OCT-embedded sections, biotinylated-isolectin B4 (1:100; Sigma, St. Louis, Mo.) was also used to visualize the structure of the vessels in the CNV lesions. Thereafter, the sections were incubated for 30 min. at room temperature with secondary antibodies (ALEXA FLUOR® 546, Molecular Probes, Eugene, Oreg.) or FITC-conjugated streptavidin (Jackson ImmunoResearch Laboratories, Inc., West Grove, Pa.), and mounted with Vectashield mounting media with 4′,6-diamino-2-phenylindole (DAPI) (Vector Laboratories, Burlingame, Calif.). Photomicrographs were taken with a digital high sensitivity camera (Hamamatsu, ORCA-ER C4742-95, Japan) thorough an upright fluorescent microscope (DM RXA; Leica, Solms, Germany). As a negative control, the primary antibodies were replaced with non-immune mouse IgG (Dako North America, Inc., Carpinteria, Calif.).

### CB Inhibition

To block CB, a specific CBD, Compound 11 described above, was used (R-tech Ueno, Ltd., Tokyo, Japan). After laser injury, the inhibitor (0.3 mg/kg BW) was administered to the animals by daily i.p. injections. As a control, some animals received the same regimen for the vehicle solution alone. Compound II has an IC₅₀ of 0.007 μM against human and 0.008 μM against rat semicarbazide-sensitive amine oxidase (SSAO), whereas its IC₅₀ against the functionally related monoamine oxidase (MAO)-A and MAO-3 is greater than 10 μM.

### Fluorescein Angiograph

Seven days after laser injury, vascular leakage from the CNV lesions was assessed using fluorescein angiography (FA), as described previously (Zambarakji et al. (2001) IOVS 42: 1553-60). Briefly, FA was performed in anesthetized animals from CBD- or vehicle-treated groups, using a digital fundus camera (Model TRC 50 IA; Topcon, Paramus, N.J.). Fluorescein injections were performed intraperitoneally (0.2 ml of 2% fluorescein; Akorn, Decatur, Ill.).

FA images were evaluated by two masked retina specialists, as previously described by Zambarakji et al. Briefly, the grading criteria were: Grade-0 lesions had no hyperfluorescence, Grade-1 lesions exhibited hyperfluorescence without leakage; Grade-IIA lesions exhibited hyperfluorescence in the early or midtransit images and late leakage; and Grade-IIB lesions showed bright hyperfluorescence in the transit images and late leakage beyond the treated areas. The Grade-IIB lesions were defined as clinically significant, as described previously

### Choroidal Flatmount Preparation

One week or two weeks after laser injury and treatment with CBD or vehicle, the size of CNV lesions was quantified using choroidal flat mounts Briefly, rats were anesthetized and perfused through the left ventricle with 20 ml PBS followed by 20 ml of 5 mg/ml fluorescein labeled dextran (FITC-dextran; MW=2×106, SIGMA) in 1% gelatin. The eyes were enucleated and fixed in 4% paraformaldehyde for 3 hours. The anterior segment and retina were removed from the eyecup Four to six relaxing radial incisions were made, and the remaining RPE-choroidal-scleral complex was flatmounted with Vectashield Mounting Medium (Vector Laboratories) and coverslipped. Pictures of the choroidal flat mounts were taken and Openlab software (Improvision, Boston, Mass.) was used to measure the magnitude of the hypedfluorescent areas corresponding to the CNV lesions. The average size of the CNV lesions was then determined and used for the evaluation.

### Quantification of the Macrophage Infiltration

At 1, 3, and 7 days after laser injury and treatment with either CBD or vehicle solution, animals were perfused with 200 ml of PBS/kg BW under deep anesthesia. Subsequently, eyes were enucleated and fixed overnight with 4% PFA, and 10 μm frozen sections of the posterior segment, including the center portion of CNV lesions (6 lesions per eye), were prepared and pre-blocked (PBS containing 10% goat serum, 0.5% gelatin, 3% BSA, and 0.2% Tween 20). The sections were incubated with mouse monoclonal antibody for ED-1, rat homologue of human CD68 (1:100; BD Pharmingen, San Diego, Calif.), and subsequently incubated with the secondary antibody (goat antimouse IgG conjugated to ALEXA FLUOR® 488, Molecular Probes). Sections were mounted with Vectashield mounting media (Vector Laboratories). The photographs of CNV lesions were taken, and the numbers of ED-1-positive cells were counted. To obtain a quantitative index of macrophage numbers in CNV lesions, an optical density plot of the selected area was generated by a histogram graphing tool in the Photoshop imageanalysis software (version 6.0: Adobe Systems, Mountain View, Calif.), as described in the literature (for example, Sakurai et al. (2003) IOVS 44: 3578-85). Image analysis was performed in a masked fashion.

### Enzyme-Linked Immunosorbent Assay for TNF-α, MCP-1 and ICAM-1

The RPE-choroid complex was carefully isolated from eyes 3 days after photocoagulation and placed in 300 μl of lysis buffer supplemented with protease inhibitors and sonicated. The lysate was centrifuged at 15,000 rpm for 15 minutes at 4° C. and the levels of TNF-α, monocyte chemotactic protein (MCP)-1, and intercellular adhesion molecule (ICAM)-1 were determined with rat TNF-α (BI) bioscience), MCP-1 (BD bioscience) and ICAM-1 (R&D Systems, Minneapolis, Minn.) enzyme-linked immunosorbent assay (ELISA) kits according to the manufacturers' protocols. Total protein concentration was determined using a Bio-Rad Protein Assay Kit (Bio-Rad Laboratories Hercules, Calif.) and dilutions of bovine serum albumin (Bio-Rad Laboratories) as standards.

### Statistical Analysis

All results are expressed as mean±SEM with n-numbers as indicated. Student's t-test was used for statistical comparison between the groups. The results of the FA gradings were compared using the chi-square test. Differences between the means were considered statistically significant when the probability values were <0.05.

### Results

### CB Expression in the Choroid and CNV

To determine whether CB is expressed in the choroid, the level of its mRNA expression was examined by RT-PCR and its protein expression was examined by immunofluorescence staining. Since choroidal tissues and RPE cells usually contain melanin, which binds to thermostable DNA polymerase and interferes with the PCR amplification, albino rats that lack melanin were used. In line with a previous study, CB mRNA was detectable in the retina under normal conditions. However, RT-PCR revealed constitutive CB mRNA expression in the RPE-choroid complex under normal conditions. Semi-quantitative analysis of the band intensity showed a 2.8-fold higher expression of CB mRNA in the RPE-choroid complex compared to that in the retinal tissues (n=4 in each group, p<0.01). In addition, immunofluorescence staining of sections from the eyes of normal animals showed the expression of CB protein in the choroid and that CB was exclusively localized in the vessels.

### Role of CB in CNV Formation

To examine whether CB contributes to CNV formation, the fundus of Brown Norway Rats was photocoagulated with and without CB blockade and the size of the CNV in flat mounts of the RPE-choroid complex was quantified. In addition, CB localization in CNV was examined by immunofluorescence staining. The staining for CB protein was co-localized with isolectin B4 staining in arborizing CNV, suggesting that vascular endothelial cells in CNV lesion also express CB. Furthermore, 7 days after laser injury, the animals treated with CBD showed a significant decrease in CNV size (14,536±2175 μm², n=7), compared with vehicle-treated animals (25,026±1586 m², n=9, p<0.01). However, fourteen days after laser injury, the CNV size in the (CBD-treated animals was not significantly different compared with the vehicle-treated controls (23,992±1437 vs. 26,681±3572 μm², n=10 and 9 eyes, respectively; p=0.5).

Fluorescent angiography showed that the incidence of the clinically significant CNV lesions, graded as IIB, was significantly decreased in CBD-treated animals (41.8%, n=12) in comparison with vehicle-treated animals (64.5%, n=11; p<0.05).

### Effect of CB Blockade on Macrophage Infiltration

To investigate whether CB inhibition affects macrophage infiltration into the CNV lesion, the numbers of ED-1 positive cells in the CNV lesions of animals with or without CB inhibition were quantified. Macrophages were recruited to the CNV lesion with a peak at day 3. In comparison, the number of accumulated macrophages at 3 days after laser injury was significantly reduced, by 41%, with the blockade of CB (n=4, p<0.05).

### Reduction of Inflammatory Molecules by CB Blockade

To investigate the mechanisms by which CB blockade suppresses CNV formation, the levels of the inflammation-associated molecules, TNF-α, MCP-1 and ICAM-1, in the RPE-choroid complex were measured with or without CNV lesions at 3 days after laser irradiation. As compared to protein levels of TNF-α (282±18 pg/mg), MCP-1 (496±38 pg/mg) and ICAM-1 (50±4 ng/mg) in the RPE-choroid complex of normal rats, the protein levels of TNF-α (395±17 pg/mg, p<0.01), MCP-1 (797±53 pg/mg, p<0.01), ICAM-1 (66±3 ng/mg, p<0.01) in the RPE-choroid complex of rats with CNV were significantly increased at 3 days after laser injury. In addition, the protein levels of TNF-α, MCP-1 and ICAM-1 were significantly reduced in the RPE-choroid complex of the laser-treated animals that received the inhibitor compared with the vehicle controls (TNF-α, 407±17 vs. 360±12 pg/mg, p<0.05; MCP-1, 969±93 vs. 662±52 pg/mg p<0.01 ICAM-1, 71±4 vs. 57±2 ng/mg, p<0.01, respectively). There was no statistical difference in the protein levels of the molecules between vehicle-treated and vehicle-untreated CNV animals (TNF-α, p=0.6; MCP-J, p=0.1, ICAM-1, p=0.3, respectively).

The experiments investigated the role of CB in the formation of CNV, an integral component of AMD. The results show constitutively higher levels of CB expression in the choroid compared to the retina using RT-PCR and immunofluorescence staining CB blockade significantly reduced the CNV size seven days after laser injury and macrophage accumulation at the peak of CNV growth, three days after laser injury. These data suggests that the reduction of the CNV formation by CB blockade may in part be due to suppression of macrophage recruitment.

CB is a mediator of leukocyte recruitment, particularly of the transmigration step. Recently, CB has been shown to play a role in acute ocular inflammation. However, whether CB plays a role in the pathogenesis of AMD was previously unknown. Since inflammatory processes can be involved in the development of AMD, the role of CB in the formation of CNV, an integral component of AMD, was investigated in the experiments described in this Example. A link between CB and angiogenesis was discovered.

In addition, constitutively higher levels of CB expression were found in the choroid as compared to the retina using RT-PCR and immunofluorescence staining. This may in part be due to the higher vascular density in the choroid compared to the retina. The constitutive expression of CB in the choroid and the retina suggests a role for CB in leukocyte extravasation in both vascular beds. This suggests that CB blockade may suppress CNV development through inhibition of inflammatory leukocyte accumulation. Indeed, CB blockade was shown to significantly reduce the CNV size 7 days after laser injury and the macrophage accumulation at the peak of CNV growth, 3 days after laser injury. This suggests that the reduction of the CNV formation by CB blockade may in part be due to suppression of macrophage recruitment. However, fourteen days after laser injury, CB inhibition did not reduce CNV size, suggesting the existence of other CB independent angiogenic mechanisms that may compensate for the antiangiogenic effect of CB inhibition seven days after late injury inhibition of one angiogenic factor may lead to up-regulation of other factors with functional overlap. A variety of cytokines, chemokines, and endothelial adhesion molecules play important roles in the pathogenesis of CNV. In the current study, the impact of CB blockade on the production levels of selected members of these inflammation-associated molecules was investigated. CB blockade significantly decreased the protein level of the inflammatory cytokine. TNF-α, in the RPE-choroid complexes with CNV. Since macrophages in CNV lesions are a source of TNF-α, it is possible that the inhibition of macrophage infiltration by CB blockade may underlie the decreased level of TNF-α in the CNV lesions Interestingly, previous studies show that TNF-α inhibition reduces CNV in an animal model. Furthermore, anti-TNF-α therapy in patients with inflammatory arthritis, who also had AMD, resulted in partial CNV regression and visual acuity improvement. The FA data in the experiments in this Example shows fewer lesions with clinically relevant leakage (Grade IIb) after CB blockade, compared with the vehicle-treated animals, which suggests that TNF-α reduction through C13 blockade could be an alternate strategy for treatment of AMD.

In addition to TNF-α, CB blockade also significantly reduced the level of potent macrophage-recruiting chemokine. MCP-1, in the RPE-choroid complex after laser injury. In vitro, TNF-α is known to stimulate RPE cells to produce MCP-1. The data in the experiments described in this Example support a model in which reduced levels of MCP-1 lead to decreased macrophage infiltration. This would cause further reduction of TNF-α release, which in turn would lead to diminished secretion of MCP-1 in RPE cells. CB blockade may thus interrupt this perpetual cascade of inflammatory events that exacerbate CNV formation at the stage of macrophage transmigration.

It was also found that CB blockade significantly reduced the expression of ICAM-1 in choroidal tissues with CNV. ICAM-1, a key endothelial adhesion molecule which regulates leukocyte recruitment, is upregulated in the RPE-choroid complex during CNV formation. Mice deficient for ICAM-1 or its counter receptor, CD18, are known to develop significantly smaller CNV lesions compared with wild-type, suggesting an important role for ICAM-1 in CNV formation. The suppressive effect of CB blockade on ICAM-1 expression, as observed in this study, is generally consistent with previous data showing that CB blockade reduces the upregulation of ICAM-1 after LPS stimulation in the retina. The reduction of ICAM-1 expression after CB blockade in laser-injured eyes may result in lower macrophage infiltration and smaller CNV lesions. Overall, CB blockade appears to effectively suppress key molecular and cellular components in a cascade leading to CNV formation. This may be achieved through inhibition of macrophage infiltration and through reduction of the levels of inflammatory cytokines, chemokines and adhesion molecules.

In summary, these results show that CB blockade with the specific inhibitor, Compound II, effectively suppresses CNV. CB inhibition also reduces macrophage recruitment to the CNV lesions and secretion of inflammatory factors such as MCP-1 and TNF-α in the choroidal tissues. The current results show that CBDs can be used in the treatment of angiogenic conditions, such as CNV associated with AMD.

### Example 3: CB Inhibition Suppresses Corneal New Vessel Growth

In this experiment, the role of CB in corneal angiogenesis and in corneal lymphangiogenesis was investigated. Specifically, the CBD, Compound 11 as described above, was administered to animal models of corneal angiogenesis and lymphangiogenesis. Results of this experiment identify CB as a molecular target in the prevention and treatment of both corneal angiogenesis and corneal lymphangiogenesis, as well as other angiogenic and lymphangiogenic conditions

### Experimental Animals

BALB/c mice were anesthetized by intraperitoneal (i.p.) injection of pentobarbital sodium (60 mg/kg). Hydron pellets (0.3 μl) containing 30 ng mouse IL-1β (401-ML, R&D Systems) were prepared and implanted into the corneas. Pellets were positioned 1 mm from the corneal limbus. Implanted eyes were treated with Bacitracin ophthalmic ointment (E. Fougera & Co.) to prevent infection.

### CB Inhibition

To block CB, mice received daily i.p. injections of a specific CBD. Compound II (R-tech Ueno Ltd., Tokyo, Japan) as described above. A daily dose of 0.3 mg/kg was administered at day 0 and continued until the sixth day after implantation. Two, four and six days after implantation, digital images of the corneal vessels were obtained and recorded using OpenLab software version 2.2.5 (Improvision Inc.) with standardized illumination and contrast and were saved onto disks. The quantitative analysis of new vessel growth in the mouse corneas was performed using Scion Image software (version 4.0.2, Scion Corp.).

### Whole-Mount Immunohistochemistry

Eyes were enucleated and fixed with 4% paraformaldehyde for one hour at 4° C. For whole-mount preparation, the corneas were exposed by removing other portions of the eye (i.e iris, sclera, retina, and conjunctiva). After washing with PBS, tissues were placed in methanol for 20 minutes Tissues were incubated overnight at 4° C. with antibodies for CD31 (1:25, 550274, BD Pharmingen, San Diego, Calif.), LYVE-1 (4 pg/ml, 103-PA50AG; RELIAtech, Germany), CB (1:40, sc-13743; Santa Cruz) or CB (1.20, HM1094; Hycult biotechnology, Netherlands) diluted in PBS containing 10% goat serum and 1% Triton X-100. Tissues were washed four times in PBS followed by incubation with FITC-conjugated goat anti-rat Ab (1:100. AP136F; Chemicon International), Alexa Fluor 647 goat anti-rabbit Ab (1:100, A21244; Invitrogen) or Alexa Fluor 647 chicken anti-goat Ab (1:100. A21469; Invitrogen) overnight at 4° C. Radial cuts were then made in the peripheral edges of the tissue to allow flat mounting on a glass slide in mounting medium (Vectashield; Vector Laboratories).

### Immunostaining

Mice were sacrificed under deep anesthesia with pentobarbital sodium (60 mg/kg i.p.). The eyes were harvested, snap-frozen in optimal cutting temperature (OCT) compound (Sakura Finetechnical) and 10 μm sections were prepared, air-dried and fixed in cold acetone for 10 min. The sections were blocked with nonfat dried-milk (M7409; Sigma) for 10 minutes and stained with anti-CD11b mAb (1.100, 550282, BD Pharmingen), anti-Gr-1 mAb (1.100, 550282, BD Pharmingen) or anti-F4/80 mAb (1:100, MCA497G: Serotec). After an overnight incubation, sections were washed and stained for 20 min with secondary Abs, FITC-conjugated goat anti-rat (1:100, AP136F; Chemicon International).

### CB Blockade Inhibits IL-1β-Induced Angogenesis

It was found that i.p. administration of a CBD significantly reduced corneal angiogenesis. In control mice exposed to IL-1β alone or IL-1β+vehicle, a significant increase in neovascularization was observed at day 6. However, in the mice treated with IL-1β+CBD, there was a significant reduction in inflammatory corneal angiogenesis. Quantitatively, the neovascular area at day 6 in the IL-1β+CB) mice was about half that of the neovascular area of the control mice exposed to IL-1β alone or IL-1β+-vehicle.

To examine the effect of CB inhibition on leukocyte infiltration, the infiltration of CD11b(+) cells was compared between corneas of animals treated with a CBD and corneas of untreated animals. The comparison indicates that infiltration of CD11b(+) cells was effectively inhibited by systemic administration of the CBD.

To examine which population of leukocytes was affected by CB blockade, the number of Gr-1(+) cells (indicative of neutrophils and macrophages) and F4/80(+) cells (indicative of monocytes and macrophages) in IL-1β-implanted corneas was examined. A comparison of the number of Gr-1(+) cells and F4/80(+) cells, respectively, appearing in IL-1β-implanted cornea with and without CB inhibition, following implantation was done. Both the number of Gr-1(+) cells and F4/80(+) cells in CBD-treated cornea were less than in vehicle-treated cornea or untreated cornea. This result is consistent with a number of studies which have suggested that leukocytes play an important role in corneal angiogenesis. Specifically, if CD11b(+) cells are a factor in corneal angiogenesis, then the mechanism by which CB blockade inhibits angiogenesis may include inhibition of CD11b(+) cells, as seen in these results.

### CB Blockade Inhibits IL-1β-Induced Lymphangiogenesis

It was found that i.p. administration of a CBD reduced corneal lymphangiogenesis. Corneal tissue samples were analyzed following induction of corneal lymphangiogenesis with IL-1β and treatment with vehicle (IL-1β+Vehicle) or CBD (IL-1β+CB inh). Anti-LYVE-1 stain identifies lymphatic vessels. It was shown that CBD reduced growth of lymphatic vessels in a lymphangiogenesis model.

### CB Expression in Non-Inflamed Versus Inflamed Corneas

CB expression in inflamed and non-inflamed corneas was also compared Immunohistochemistry showed that CB was expressed in blood vessels in both inflamed and non-inflamed corneas (with and without IL-1β implantation) Samples were stained with anti-CD31 to identify endothelial cells in blood vessels. Additional samples were stained with anti-CB to identify the presence of CB. The images were combined and indicate that CB is expressed on quiescent blood vessels. Corneal tissue from corneas treated with IL-1β to induce angiogenesis was reviewed. Samples were stained with anti-CD31 to identify endothelial cells in blood vessels Other samples were stained with anti-CB to identify the presence of CB. The images of the two sets of samples was combined, and indicates that CB is expressed on angiogenic blood vessels.

However, CB did not appear to be expressed in lymphatic vessels in un-inflamed cornea (no IL-1β implantation). Untreated corneal tissue (no IL-1β treatment) was also reviewed. Samples were stained with anti-CB to identify the presence of CB. Additional samples were stained with anti-LYVE-1 to identify lymphatic vessels. The images, when merged indicate that CB is not expressed on quiescent lymphatic vessels.

These results show that CB blockade with the specific inhibitor, Compound II, effectively suppresses corneal angiogenesis as compared untreated controls. CB inhibition also reduces CD11b(+) cells in the cornea and limbus.

These results also show that CB blockade with the specific inhibitor, Compound II, effectively suppresses corneal lymphangiogenesis as compared untreated controls. Accordingly, the current results show that CBDs can be used in the treatment of corneal angiogenesis and in the treatment of corneal lymphangiogenesis, as well as other angiogenic and lymphangiogenic conditions.

### Example 4: CB Inhibition Suppresses Metastatic Tumor Growth

The following experiment describes a method for observing the ability of a CBD to suppress metastatic tumor growth.

### Method

Animals with a Lewis lung carcinoma tumor between 600-1200 mm³ in size are sacrificed and the skin overlying the tumor is cleaned with betadine and ethanol. In a laminar flow hood, the tumor tissue is excised under aseptic conditions. A suspension of tumor cells in 0.9% normal saline is made by passage of viable tumor tissue through a sieve and a series of sequentially smaller hypodermic needles of diameter 22- to 30-gauge. The final concentration is adjusted to 1×10⁷ cells/ml and the suspension is placed on ice. After the site is cleaned with ethanol, the subcutaneous dorsa of mice in the proximal midline are injected with 1×10⁶ tumor cells in 0.1 ml of saline.

When tumors reach 1500 mm³ in size, the tumors are surgically removed from the mice. The incision is closed with simple interrupted sutures. From the day of operation, mice receive daily injections of a CBD or a saline control. When the control mice become sick from metastatic disease (i.e, after 13 days of treatment), all mice are sacrificed and autopsied. Lung surface metastases are counted by means of a stereomicroscope at 4× magnification.

### Expected Results

It is expected that mice treated with the CBD as compared to control mice treated with saline show significantly diminished metastasized tumor growth in the lungs The following experiment describes a method for observing the ability of a CBD to suppress primary tumor growth.

### Methods

Mice are implanted with Lewis lung carcinomas as described in Example 3. Tumors are measured with a dial-caliper and tumor volumes are determined, and the ratio of treated to control tumor volume (T/C) is determined for the last time point. After tumor volume is 100-200 mm³ (0.5-1% of body weight), mice are randomized into two groups. One group receives the CBD injected once daily. The other group receives comparable injections of the vehicle alone. The experiments are terminated and mice are sacrificed and autopsied when the control mice begin to die.

### Expected Results

It is expected that the growth of Lewis lung carcinoma primary tumors is inhibited by the administration of the CBD as compared to the saline control.

### Example 5: Localization of CB in the Human Eye

To further understand the role of CB in angiogenic disorders, such as ocular angiogenic disorders, the expression of CB in the human eye was investigated. This example shows that, in the human, CB is localized to areas consistent with the data shown in Examples 1 and 2 as well as its role as a therapeutic target for ocular angiogenic conditions described herein Briefly, five micrometer thick sections were generated from human ocular tissues embedded in paraffin. CB localization was investigated by immunohistochemistry Sections were incubated overnight with primary monoclonal antibodies against CB (5 pg/ml), smooth muscle actin (1 pg/ml), CD31, or isotype-matched IgG at 4° C. Subsequently, a secondary monoclonal antibody was used for 30 minutes at room temperature, followed by use of the Dako Envision+HRP (AEC) System (available from Dako North America, Inc., Carpenteria, Calif.) for signal detection. The stained sections were examined using light microscopy, and the signal intensity was quantified by two masked evaluators and graded into four discrete categories.

In all examined ocular tissues, CB staining was confined to the vasculature. CB labeling showed the highest intensity in both arteries and veins of neuronal tissues, retina and optic nerve, and the lowest intensity in the iris vasculature. Scleral and choroidal vessels showed moderate staining for CB. CB intensity was significantly higher in the arteries compared to veins. Furthermore, CB staining in arteries co-localized with SM-actin staining, suggesting expression of CB in smooth muscle cells or, potentially, pericytes.

Immunohistochemistry revealed constitutive expression of CB in human ocular tissues. CB expression is exclusive to the vasculature with arteries showing significantly higher expression than veins. Furthermore, CB expression in the ocular vasculature is heterogeneous, with the vessels of the optic nerve and the retina showing highest expressions. These results suggest CB is a relevant molecule in ocular vascular and inflammatory diseases in humans.

a. Methods

### Tissue Samples

Paraffin-embedded blocks of normal human ocular tissues were obtained from the Massachusetts Eye and Ear Infirmary's (MEEI) stored archives of samples. All materials were used in accordance with the protocol approved by the Institutional Review Board (IRB) of the MEEI and in accordance with the Declaration of Helsinki.

### Immunohistochemistry

CB tissue localization was examined in paraffin-embedded sections of human eyes. The slides were dewaxed and hydrated through exposure with graded alcohols (100% then 95%) followed by water. Endogenous peroxidase activity was then blocked by placing the sections in 0.3% hydrogen peroxide (Sigma Aldrich, St. Louis, Mo., US) for 15 minutes, and non-specific binding was blocked by subsequently placing the sections in 10% normal goat serum (Invitrogen, CA) for 1 hour. Subsequently, the sections were reacted with primary monoclonal antibodies (mAb) against either CB (5 pg/ml; BD Biosciences, Franklin Lakes, N.J.), endothelial CD31 (Dako North America, Inc., Carpinteria, Calif.) or smooth muscle actin (1 pg/ml; Sigma, St Louis, Mo.) at 4° C. overnight. For CD31 staining, deparaffinized sections were heated in a water bath at 97° C. for 10 minutes.

Thereafter, the sections were incubated for 30 minutes at room temperature with Envision system secondary antibodies against mouse IgG (Dako North America, Inc., Carpinteria, Calif.). For signal detection, the Dako Envision+HRP (AEC) System was used according to the manufacturer's protocol. Finally, sections were counterstained with hematoxylin. Photomicrographs were taken with a digital high sensitivity camera (Hamamatsu, ORCA-ER C4742-95, Japan). As a negative control, the primary antibodies were replaced with non-immune mouse IgG (Dako North America. Inc., Carpinteria, Calif.).

### Data and Statistical Analysis

Histological sections were examined under light microscopy and graded by two independent experimenters CB signal intensity was judged as: no (“−”), moderate (“+”), and strong (“++”) staining. To compare the results from different groups, the grades given by the observers were averaged for each eye and plotted as 0, 1 and 2, respectively. For statistical analysis, the results were divided into two groups (0 or higher). A Chi-square test was used to calculate the degree of confidence with which the data supports the null hypothesis. Probability values (p) less than 0.05 were considered statistically significant.

b. Results

### Exclusive Expression of CB in the Vasculature of the Human Eye

To determine CB expression in the human eye, immunohistochemistry was performed on normal human ocular tissues (n=7). In various ocular tissues, CB specific signal was almost exclusively confined to the vasculature as compared to nonimmune isotype control. Particularly, CB was observed in the inner and medial layers, but not the outer adventitial layer, of the main branches of the ophthalmic artery. In contrast, small capillaries did not show CB expression. Outside of the vessels, CB expression also was observed in the smooth muscle cells of the ciliary body while no CB staining was observed in the retinal pigment epithelium (RPE) layer of any of the eyes.

### CB Distribution in Normal Human Ocular Tissues

To compare the vascular CB expression in different ocular tissues, CB signal intensity was quantified by grading. No appreciable staining for CB was observed in the iris vessels, both arteries and veins (n=4). Compared with the iris arteries, arteries of the choroidal (n=6) and scleral (n=7) tissues (n=7) showed significantly higher CB staining (p<0.05), and arteries of neuronal tissues, the retina (n=6) and optic nerve (n=7), showed the most prominent staining (p<0.05 and p<0.01, respectively). In contrast, no significant difference was observed in venular CB expression of all groups (p>0.1).

CB expression also was compared between arteries and veins. CB expression was significantly higher in arteries than veins in all examined tissues (p<0.05), except for the iris vessels.

### Localization of CB to Both Vascular Endothelial and Smooth Muscle Cells

To further investigate the cellular distribution of CB, co-immunostaining of CD31, a marker for endothelial cells, and sm-actin, a marker for smooth muscle cells, was performed. In line with previous studies in various other human tissues (Jaakkola, K. et al. (1999) AM J PATHOL 155:1953-1965) in the eye, CB co-localized both in endothelial and smooth muscle cells.

Distribution pattern of CB in human ocular tissues was determined. In the eye, CB is exclusively expressed in the vasculature. Arteries show significantly higher levels of CB staining than veins, suggesting a specialized role for this molecule in diseases with primary arterial involvement. The difference between arterial and venous expression may be relevant in the pathogenesis of diabetic retinopathy, where capillary non-perfusion, due to leukocyte plugging at the capillary entrance has been postulated as an important component (Miyamoto et al. (1999) PNAS USA 96:10836-10841; Miyamoto el al. (1999) Semin Ophthalmol 14:233-239; Schroder (1991) AM J Pathol 139:81-100). Most adhesion molecules, such as ICAM-1 or P-selectin, which lead to leukocyte adhesion in postcapillary venules, would not sufficiently explain this phenomenon (Miyamoto et al PNAS USA, supra). Furthermore, the higher expression of CB in arteries together with the specialized role of this molecule for leukocyte transmigration confirms this molecule as a target in ocular diseases, such as ocular angiogenic conditions.

These studies also indicate that in addition to the endothelium, smooth muscle cells also express CB. Since arteries have both endothelial and smooth muscle cells, while veins have only endothelial cells, this might in part explain the higher level of CB expression in arteries compared to veins. Furthermore, heterogeneity in the vascular expression of CB was found within the various regions of the eye. While vessels of the optic nerve head expressed highest amounts of the molecule, the iris vessels did not show detectable expression. The broad expression of CB in the posterior section of the eye suggests an involvement of the molecule in ocular diseases, such as age-related macular degeneration and diabetic retinopathy in humans. The experiments in this Example show constitutive expression of CB in humans, show its presence in human tissues consistent with its role as a therapeutic target for ocular angiogenic conditions described herein, and confirm its role in human angiogenic conditions, such as ocular angiogenic conditions.

### EQUIVALENTS

The embodiments may have other specific forms without departing from the spirit or essential characteristics thereof. The foregoing embodiments are therefore to be considered in all respects illustrative rather than limiting on the invention described herein.

### INCORPORATION BY REFERENCE

The entire disclosure of each of the patent documents and scientific publications disclosed hereinabove is expressly incorporated herein by reference for all purposes.

### REFERENCES

[1] Klein, R., et al. Diabetes in America (1995) National Institute of Health 293-338
[2] Tien Yin Wong, et al. Am J Ophthalmol. (2006) March; 141(3): 446-455
[3] Ryan Lee, et al. Eye Vis (London). (2015); 2: 17.
[4] Bressler, et al. (1988) Surv. Ophthalmol. 32, 375-413,
[5] Guyer, et al. (1986) Arch. Ophthalmol. 104, 702-705
[6] Hyman, et al. (1983) Am. J. Epidemiol. 188, 816-824
[7] Klein & Klein (1982) Arch. Ophthalmol. 100, 571-573
[8] Leibowitz, et al. (1980) Surv. Ophthalmol. 24, 335-610
[9] Yau, et al. (2012) March; 35(3): 556-564
[10] Ferris, et al. (1999) N Engl J Med. 341, 667-678
[11] Zhang, et al. (2014) Cell Biosci. 4, 27
[12] Klein, et al (2007) Ophthalmic Epidemiol. 14, 179-183
[13] Klein, et al (1984) Arch Ophthalmol 102:527-532
[14] Klein, et al (1992) Ophthalmology. 99:58-62
[15] The Diabetes Control and Complications Trial Research Group (1993) N Engl J Med 329 977-986
[16] Zhang, X., et al, (2010) JAMA 304:649-656
[17] Shaw, J. E., et al, (2010) Diabetes Res Clin Pract 87:4-14
[18] Rema, M., et al., (2005) Invest Ophthalmol Vis Sci; 46:2328-2333
[18] Wong, T. Y., et al. (2006) Am J Ophthalmol; 141:446-455
[19] Kempen, J. H., et al (2004) Arch Ophthalmol; 122:552-563
[20] Klein, R., et al (1984) Arch Ophthalmol; 102:527-532
[21] Mitchell, P., et al (1998) Ophthalmology; 105:406-411
[22] Varma, R., et al (2004) Ophthalmology; 111:1298-1306
[23] Haffner, S., (1993) Ann Epidemiol; 3:2-8
[24] Harris, M. I., et al (1998) Diabetes Care; 21:1230-1235
[25] Liew, G., et al (2009) Int Ophthalmol Clin; 49:35-52
[26] Harris, E. L., et al (1993) Diabetes Care; 16:748-754
[27] Roy, M. S., et al (2004) Arch Ophthalmol; 118:105-115
[28] Rema, M., et al (2005) I. Invest Ophthalmol Vis Sci; 46:2328-2333
[29] Yang, et al (2010) N Engl J Med; 362:1090-1101
[30] Geraldes (2010) Circ Res; 106(8): 1319-1331.
[40] S. Y. Cohen, et al. Br J Ophthalmol. 2007 September; 91(9): 1173-1176.
[41] Jing-Hong Chang, et al. Surv Ophthalmol. 2012 September; 57(5): 415-429
[42] Jessica F. Yang, et al. Surv Ophthalmol. 2016 May-June; 61(3): 272-296
[43] Shintaro Nakao, et al. J Ophthalmol. 2012; 2012: 783163
[44] Stevenson, et al. Arch Ophthalmol. 2012 January; 130(1): 90-100
[45] Peach, et al. Int J Mol Sci. 2018 April; 19(4)
[46] Hyeong Gon Yu, et al. Invest Ophthalmol Vis Sci. 2008 June; 49(6): 2599-2605.
[47] Cheung N., Mitchell P., Wong, T. Y., Diabetic retinopathy. Lancet. 2010 Jul. 10; 376(9735): 124-36.
[48] Bourne, R. R., Stevens, G. A., White, R. A., Smith, J. L., Flaxman, S. R., Price, H., Jonas, J. B., Keeffe, J., Leasher, J., Naidoo, K., Pesudovs, K., Resnikoff, S., Taylor, H. R., Vision Loss Expert Group. Causes of vision loss worldwide, 1990-2010: a systematic analysis. Lancet Glob Health. 2013 December; 1(6):e339-49.
[49] Yau, J. W., Rogers, S. L., Kawasaki, R., Lamoureux, E. L., Kowalski, J. W., Bek, T., Chen, S. J., Dekker, J. M., Fletcher, A., Grauslund, J., Haffner, S., Hamman, R. F., Ikram, M. K., Kayama, T., Klein, B. E., Klein, R., Krishnaiah, S., Mayurasakorn, K., O'Hare, J. P., Orchard, T. J., Porta, M., Rema, M., Roy, M. S., Sharma, T., Shaw, J., Taylor, H., Tielsch, J. M., Varma, R., Wang, J. J., Wang, N., West, S, Xu, L., Yasuda, M., Zhang, X., Mitchell, P., Wong, T. Y., Meta-Analysis for Eye Disease (META-EYE) Study Group. Global prevalence and major risk factors of diabetic retinopathy. Diabetes Care. 2012 March; 35(3):556-64.
[50] Lightman, S., Towler, H. M., Diabetic retinopathy. Clin Cornerstone. 2003; 5(2):12-21.
[51] Tong, L., Vernon, S. A., Kiel, W., Sung, V., Orr GM Association of macular involvement with proliferative retinopathy in Type 2 diabetes. Diabet Med. 2001 May; 18(5):388-94.
[52] Risk of cardiovascular diseases is increased even with mild diabetic retinopathy: the Japan Diabetes Complications Study Kawasaki, R., Tanaka, S., Tanaka, S., Abe, S., Sone, H., Yokote, K., Ishibashi, S., Katayama, S., Ohashi, Y., Akanuma, Y., Yamada, N., Yamashita, H., Japan Diabetes Complications Study Group. Ophthalmology. 2013 March; 120(3):574-582.
[53] Diabetic retinopathy in predicting diabetic nephropathy in patients with type 2 diabetes and renal disease: a meta-analysis. He F, Xia X, Wu X F, Yu X Q, Huang F X Diabetologia. 2013 March; 56(3):457-66.
[54] Shaw J E, Sicree R A, Zimmet P Z. Global estimates of the prevalence of diabetes for 2010 and 2030. Diabetes Res Clin Pract 2010; 87:4-14 [PubMed] [Google Scholar]
[55] Kempen, J. H., O'Colmain, B. J., Leske, M. C., et al.; Eye Diseases Prevalence Research Group, The prevalence of diabetic retinopathy among adults in the United States. Arch Ophthalmol 2004; 122:552-563
[56] Voraporn Chaikitmongkol, Onnisa Nanegrungsunk, Direk Patikulsila, Paisan Ruamviboonsuk, Neil M. Bressler Repeatability and Agreement of Visual Acuity Using the ETDRS Number Chart, Landolt C Chart, or ETDRS Alphabet Chart in Eyes With or Without Sight-Threatening Diseases JAMA Ophthalmol. 2018 March; 136(3): 286-290
[57] Ronald E. Gangnon, Matthew D. Davis, Larry D. Hubbard, Lloyd M. Aiello, Emily Y. Chew, Frederick L. Ferris, III, Marian R. Fisher, and the Early Treatment Diabetic Retinopathy Study Research Group A Severity Scale for Diabetic Macular Edema Developed from ETDRS Data Invest Ophthalmol Vis Sci. 2008 November; 49(11): 5041-5047.

1. A composition comprising Cannabidiol (CBD) for use in the treatment of angiogenesis in proliferative diabetic retinopathy in Diabetes.

2. The composition according to claim 1, wherein the proliferative diabetic retinopathy is angiogenic proliferative diabetic retinopathy with visual impairment.

3. The composition according to claim 1, wherein the proliferative diabetic retinopathy is treatment-resistant.

4. The composition of claim 1, wherein the CBD is for use in combination with one or more concomitant anti-angiogenic drugs (AAD).

5. The composition of claim 1, wherein the CBD is present as a highly purified extract of cannabis which comprises at least 98% (w/w) CBD.

6. The composition according to claim 5 wherein the extract comprises less than 0.15% THC.

7. The composition of claim 5 wherein the extract further comprises up to 1% CBDV.

8. The composition of claim 1, where in the CBD is present as a synthetic compound.

9. The composition according to claim 4, wherein the one or more AAD is selected from the group consisting of: dietary modification, improved glycemic control, insulin, intraocular steroids (triamcinolone), oral hypoglycemic drugs (four classes including sulfonylureas, metformin, thiazolidinediones, and alpha-glucosidase inhibitor) and/or anti vascular endothelial growth factor (anti-VEGF) agents selected from the group consisting of repaglanide, natiglinide, metformin, rosiglitazone, pioglitazone, pegaptanid, ranibizumab, bevacizumab, afibercept, verteprotin, Lapatinib, Sorafenib, Sunitinib, Axitinib, Pazopanib, pan retinal photocoagulation (PRP), focal photocoagulation, and pars plana vitrectomy.

10. The composition of claim 4, wherein the number of different AAD that are used in combination with the CBD is reduced.

11. The composition of claim 4, wherein the dose of AAD that are used in combination with the CBD is reduced.

12. The composition of claim 1, wherein the dose of CBD is greater than 5 mg/kg/day.

13. A method of treating angiogenesis in proliferative diabetic retinopathy comprising administering composition of claim 1 to a subject.

14. The composition of claim 1 for the treatment of diabetes characterized by focal seizures, said composition further comprising a solvent, a co-solvent, a sweetener, and a flavoring.

15. The composition according to claim 14, wherein the solvent is sesame oil.

16. The composition according to claim 14, wherein the co-solvent is ethanol.

17. The composition according to claim 14, wherein the sweetener is sucralose.

18. The composition according to claim 14, wherein the flavoring is strawberry flavor.

19. The composition according to claim 14, wherein the CBD is present at a concentration of between 25/mg/ml and 100 mg/ml.

20. The composition of claim 14, which comprises CBD at a concentration of between 25 to 100 mg/ml, ethanol at a concentration of 79 mg/ml, sucralose at a concentration of 0.5 mg/ml, strawberry flavoring at a concentration of 0.2 mg/ml and sesame q.s. to 1.0 ml.

21. A method for treating a lymphangiogenic condition, the method comprising: administering a composition comprising Cannabidiol (CBD) to a subject in an amount sufficient to inhibit lymphangiogenesis.

22. The method of claim 21, further comprising performing photodynamic therapy.

23. The method of claim 21, further comprising administering a VEGF inhibitor.

24. The method of claim 21, wherein the composition is administered locally.

25. The method of claim 21, wherein the condition is selected from the group consisting of scar formation, tissue repair, wound healing, rheumatoid arthritis, and organ transplantation.

26. The method of claim 21, wherein inhibition of lymphangiogenesis comprises lymph vessel regression or inhibition of lymph vessel formation.

27. The method of claim 21, wherein the lymphangiogenic condition comprises corneal lymphangeogenesis and the CBD is administered to the subject in an amount sufficient to inhibit corneal lymphangiogenesis.

28. The composition of claim 1, wherein the CBD is a cannabinoid S, L, or R isomer.

29. (canceled)
